(19) United States
(12) Patent Application Publication
(10) Pub. No.: US 2022/0013197 A1
(43) Pub. Date: Jan. 13, 2022

(54) METHOD FOR IDENTIFYING AN UNKNOWN BIOLOGICAL SAMPLE FROM MULTIPLE ATTRIBUTES

(71) Applicant: Agency for Science, Technology and Research, Singapore (SG)

(72) Inventors: Ian Walsh, Singapore (SG); Katherine Louisa Wongtrakulkish, Singapore (SG); Terry Nguyen-Khuong, Singapore (SG); Pauline Mary Rudd, Singapore (SG)

(21) Appl. No.: 17/295,418

(22) PCT Filed: Nov. 20, 2019
(86) PCT No.: PCT/SG2019/050567

§ 371 Date: May 19, 2021

(30) Foreign Application Priority Data
Nov. 23, 2018 (SG) 10201810500R

(51) Int. Cl. G16C 20/70; G16C 20/20; G06N 20/20; G06N 3/08

(52) U.S. Cl. CPC G16C 20/70; G06N 3/08; G06N 20/20; G16C 20/20

(57) ABSTRACT

A method for identifying an unknown biological sample (e.g. a glycan, an antibody, a metabolite) is disclosed. The method comprises: receiving more than two sample measurements for the unknown biological sample, calculating a sample point in a two-dimensional plot from the more than two sample measurements for the unknown biological sample and identifying the unknown biological sample by comparing the sample point against the plurality of reference points in the two-dimensional plot. The two-dimensional plot includes a plurality of stored reference points corresponding to respective known biological compounds. Each reference point is calculated from a plurality of reference measurements for more than two attributes of the corresponding known biological compound (e.g. by performing principal component analysis on the plurality of reference measurements), with each attribute being different from another attribute. Each reference measurement may be obtained experimentally (e.g. by liquid chromatography, mass spectrometry, tandem mass spectrometry, ion mobility spectrometry) or by a machine learning algorithm.

### CROSS-REFERENCE TO RELATED APPLICATION

This application claims the benefit of priority of Singapore application No. 10201810500R filed Nov. 23, 2018, the contents of it being hereby incorporated by reference in its entirety for all purposes.

### TECHNICAL FIELD

Various aspects of this disclosure relate to a method for identifying an unknown biological sample. Various aspects of this disclosure relate to a computer program product and an apparatus for implementing a method for identifying an unknown biological sample.

### BACKGROUND

Biological compounds include organic compounds associated with various life processes. One type of biological compounds includes glycans which are the carbohydrate portions of glycoconjugates, such as glycoproteins and glycolipids. Glycans are involved in many physiological and pathological processes. Therefore, understanding the glycan structures and roles in these processes can help in the design of drugs and hence, the treatment of various disease states.

Glycosphingolipids (GSLs) are a type of glycolipids including glycans. In particular, GSLs are amphipathic lipid molecules most commonly found in the cell membrane. Each GSL typically includes a hydrophilic glycan head-group attached to a hydrophobic ceramide/lipid tail. The regulation of GSL biosynthesis and metabolic pathways helps to ensure that their biological functions, including their roles in cell growth, signal transduction, and cell identity establishment and maintenance, are properly carried out. Heterogeneity in both the ceramide tails and glycan head-groups can result in a large number of GSL species, with over 500 characterised so far, and with much of the GSLs' biological functions determined by their glycan head-groups. In particular, the glycan head-groups of GSLs found in the cell membrane bilayer can alter in response to different cellular states, external stimuli and diseases, making them potential markers for cellular disease states and potential targets for drugs.

The glycan head-groups (or in short, glycans) of GSLs share a high degree of compositional similarity, but display a high degree of structural heterogeneity due to differences in their monosaccharide sequences, linkages, anomericity and branching. Further complexity can arise through monosaccharide modification of the glycans with substituents such as sulfate, phosphate and acetate. The analytical challenge in GSL glycomics lies in unearthing the structural complexities of the GSLs to gain a more comprehensive understanding of altered GSL processing pathways and the role of the glycans in cell functions and diseases. By performing comprehensive analyses of the glycan structures, markers for certain cellular disease states can be identified.

Workflows for analyzing GSLs' glycans typically involve releasing the glycans from a mixture of glycoconjugates (e.g. glycoproteins) or a specific glycoconjugate (e.g. glycoprotein), injecting the released glycans into analytical instrumentation and performing data analysis to identify the glycans. The analytical instrumentation may perform techniques such as liquid chromatography (LC), mass spectrometry (MS) and tandem mass spectrometry (MSⁿ), where each of these techniques can be used to obtain measurements for a particular attribute (e.g. mass-to-charge ratio (m/z), glucose unit (GU)) of a glycan to identify the glycan. The measurements obtained are indicative of the structure and behavior of the glycans when the techniques are performed. For example, a measurement for the m/z (m/z value) of a glycan may be an indication of the glycan's mass, and a measurement for the GU (GU value) of a glycan may be an indication of the retention time of the glycan during LC, with the retention time normalized against an established standard such as the separation of a dextran ladder (a homopolymer containing incremental glucose polymers) to account for varying experimental conditions during LC.

FIG. 1 shows a flow diagram of a conventional workflow 100 for identifying glycans using LC and MS. As shown in FIG. 1, a biological sample 102 (that may include one or more glycans released from a mixture of glycoproteins) may be injected into a LC instrument 104 and a MS instrument 106, and a LC-MS data analysis may be performed on the biological sample 102 (at 108). The biological sample 102 may also be injected into the LC instrument 104 and an MSⁿ instrument 110 and a LC-MSⁿ data analysis may be performed (at 112) on the biological sample 102. The biological sample 102 may also be injected into both the instruments 106, 110 and an MSⁿ data analysis may be performed (at 114) on the biological sample 102. As shown in FIG. 1, the biological sample 102 may be identified as containing a glycan having a structure 116 using each of the data analyses performed at 108, 112, and 114.

One technique using LC and MS to identify released, fluorescently labelled glycans is the hydrophilic interaction ultra-high performance liquid chromatography with fluorescence coupled with electrospray ionisation mass spectrometry technique (HILIC-UPLC-FLD ESI-MS). In this technique, an elution profile of the glycans is obtained and standardised using a dextran glucose homopolymer. This standardised elution profile contains multiple chromatographic peaks corresponding to respective glycans (in other words, multiple glycan peaks), and each glycan peak in the profile is associated with a GU value. The GU value of each glycan represents its normalized retention time in the HILIC-UPLC-FLD ESI-MS technique, and is related to the hydrophilicity of the glycan. The technique provides relative quantitation information based on fluorescence detection and allows users to compare experimentally derived GU values of an unknown/unidentified glycan against libraries of known/identified glycans with known GU values (such as those contained in the GlycoStore database) to identify the unknown glycan. The MS technique further produces m/z values which can be used to derive mass values of the glycans. Automated glycan assignment can then be performed by mass and GU matching of experimental mass and GU values to known mass and GU values of known glycans.

However, due to high glycan heterogeneity, GU values of isomeric structures can be highly similar, and hence, multiple glycans may elute in a single chromatographic peak with a similar GU value in complex samples. This can lead to ambiguity in structural assignments when using LC-MS techniques. FIGS. 2A to 2D show how ambiguity in structural assignments may arise. In particular, FIG. 2A shows an elution profile 200 obtained after performing LC on a biological sample including a monoclonal antibody, where the elution profile 200 shows intensities of signals (Signal [EU]) for the analytes in the biological sample as a function of their retention times in minutes (min). The retention time of each peak in the elution profile 200 may be normalized to a GU value. In FIG. 2A, the GU value of each peak is shown in a box (e.g. box 200a) connected by a line to the peak. FIG. 2B shows a plot 204 illustrating results obtained after performing MS on the analyte to which the peak 202 in FIG. 2A corresponds. In particular, the plot of FIG. 2B shows ion signal intensities (Intensity [Counts]) as a function of observed mass values (in the form of m/z). FIGS. 2C and 2D show two isomers 210, 212 that have similar m/z values and GU values. In particular, the isomer 210 in FIG. 2C has a GU value of 7.5719, a m/z value (charge=+1) of 1907.7261 and a m/z value (charge=+2) of 954.3667; whereas, the isomer 212 in FIG. 2D has a GU value of 7.4733, a m/z value (charge=+1) of 1907.7261 and a m/z value (charge=+2) of 954.3667. As shown in FIG. 2D, the isomer 212 includes an additional a-galactose branch 212a as compared to the isomer 210, but both the isomers 210, 212 correspond to the same peaks (peaks 202, 206, 208) in FIGS. 2A and 2B. Therefore, when comparing the LC-MS results against a library of glycans with known GU and m/z values, the presence of the peaks 202, 206, 208 in FIGS. 2A and 2B may indicate either the presence of the isomer 210 or the presence of the isomer 212. As a result, ambiguity in structural assignment arises.

To address the ambiguity in structural assignments, an ion mobility mass spectrometry technique (IM-MS) may be used to improve the identification of closely related analytes such as isomeric or isobaric glycans. This technique distinguishes different glycans based on their three-dimensional shapes, sizes and charges. In particular, the technique utilises the separation of gas-phase ions in a drift tube, where ions move under an electric field in a buffer gas. The time taken for a glycan to travel through the drift tube can be used to calculate Collision Cross Section (CCS) values using the Mason-Schamp equation. CCS values can be utilised as glycan identifiers and, in addition to GU and m/z values, can increase the confidence level in the matching of experimental data of a glycan to a reference database. Therefore, using IM as an additional level of separation can aid the characterization of closely-related or isometric structures through the generation of glycan CCS identifiers.

Prior art approaches generally use either one or at most two attributes to identify unknown glycans, and incomplete assignment and characterization of glycans often occur, especially when the glycans have isometric structures. To resolve this, further targeted experiments may be performed but such experiments can considerably slow down the glycan identification process.

### SUMMARY

Various embodiments may provide a method for identifying an unknown biological sample. The method may include receiving more than two sample measurements for the unknown biological sample, calculating a sample point in a two-dimensional plot from the more than two sample measurements for the unknown biological sample, and identifying the unknown biological sample by comparing the sample point against the plurality of reference points in the two-dimensional plot. The two-dimensional plot may include a plurality of stored reference points corresponding to respective known biological compounds and each reference point may be calculated from a plurality of reference measurements for more than two attributes of the corresponding known biological compound, each attribute being different from another attribute. Each sample measurement may be for an attribute of the unknown biological sample.

Various embodiments may provide a computer program product including computer-readable instructions that implement an application for identifying an unknown biological sample. The computer program product may be configured to be executed on one or more computing devices, each having one or more processors. The application may be configured to provide a two-dimensional plot including a plurality of stored reference points corresponding to respective known biological compounds and each reference point may be calculated from a plurality of reference measurements for more than two attributes of the corresponding known biological compound, each attribute being different from another attribute. The application may include instructions for: receiving more than two sample measurements for the unknown biological sample, each sample measurement for an attribute of the unknown biological sample; calculating a sample point in a two-dimensional plot from the more than two sample measurements for the unknown biological sample; and identifying the unknown biological sample by comparing the sample point against the plurality of reference points in the two-dimensional plot.

Various embodiments may provide a kit including an extraction device for extracting an unknown biological sample; at least one experimental device for determining sample measurements for the extracted unknown biological sample; and a computing device configured to execute the above computer program product.

Various embodiments may provide an apparatus including: a memory; and at least one processor coupled to the memory and configured to: receive more than two sample measurements for the unknown biological sample, calculate a sample point in a two-dimensional plot from the more than two sample measurements for the unknown biological sample; and identify the unknown biological sample by comparing the sample point against the plurality of reference points in the two-dimensional plot. The two-dimensional plot may include a plurality of stored reference points corresponding to respective known biological compounds and each reference point may be calculated from a plurality of reference measurements for more than two attributes of the corresponding known biological compound, each attribute being different from another attribute. Each sample measurement may be for an attribute of the unknown biological sample.

### BRIEF DESCRIPTION OF THE DRAWINGS

The invention will be better understood with reference to the detailed description when considered in conjunction with the non-limiting examples and the accompanying drawings, in which:

FIG. 1 shows a flow diagram of a conventional workflow for identifying glycans using liquid chromatography and mass spectrometry;

FIGS. 2A and 2B show results obtained after performing liquid chromatography and mass spectrometry on a biological sample, and FIGS. 2C and 2D show isomeric structures present in the biological sample;

FIG. 3 shows a conceptual diagram of a system for identifying an unknown biological sample according to various embodiments;

FIG. 4 shows a flow diagram of a method implemented by the system of FIG. 3 according to various embodiments;

FIG. 5 shows a flow diagram of forming a two-dimensional plot in the method of FIG. 4 according to various embodiments;

FIG. 6 shows an example workflow to obtain reference measurements in the method of FIG. 4;

FIG. 7 shows an example workflow to form an experimental library and an in silico library in the method of FIG. 4;

FIG. 8 shows an example workflow to form a two-dimensional plot in the method of FIG. 4;

FIG. 9 shows an example workflow of updating the experimental library and the in silico library of FIG. 7;

FIG. 10 shows an example workflow of calculating a sample point for an unknown biological sample and identifying the unknown biological sample in the method of FIG. 4;

FIG. 11 shows an example workflow for the method of FIG. 4 that may include forming and using a plurality of two-dimensional plots formed with different numbers of attributes;

FIG. 12 shows an example of a hardware implementation for an apparatus that may implement the system of FIG. 3 and the method of FIG. 4;

FIG. 13 shows results from an example implementation of the method of FIG. 4 to identify an unknown glycan;

FIGS. 14A-14D show results for glycans obtained with and without a partitioning process on extracted GSLs;

FIG. 15 shows results obtained for glycans released using different digestion conditions; FIGS. 16A and 16B show results obtained for procainamide-labelled and 2-AB labelled pentassacharide samples;

FIG. 17 shows a plot illustrating percentages of correctly identified glycans when different numbers of attributes of the glycans are used for the identification process;

FIG. 18 shows a Pearson correlation analysis of different attributes of biological samples;

FIG. 19A shows a plot illustrating percentages of correctly identified glycans when different numbers of attributes of the glycans are used for the identification process and when a reduced library including only isomeric structures is used, and FIG. 19B shows a visualization of the assignment of the unknown glycans to the library glycans for the identification process when three attributes are used;

FIGS. 20A and 20B show plots with points representing different glycans with the points in the plot of FIG. 20A formed from two attributes and the points in the plot of FIG. 20B formed from more than two attributes;

FIGS. 21A to 21K show plots illustrating regression curves indicating probabilities of correctly identifying unknown glycans given distances calculated for the unknown glycans when different combinations of attributes are used;

FIG. 22A shows a Venn diagram illustrating a qualitative comparison of glycans detected from breast cancer cells, FIG. 22B shows a clustering analysis of LC-FLD peak average relative abundances of peaks commonly detected in breast cancer cells and FIG. 22C shows a clustering analysis of glycomes based on the presence/absence of glycans in a breast cancer cell;

FIG. 23 shows an average relative glycan abundance of glycans detected in breast cancer cells; and

FIG. 24 shows a plot illustrating reference measurements for attributes in an experimental library.

### DETAILED DESCRIPTION

Aspects of the present invention and certain features, advantages, and details thereof, are explained more fully below with reference to the non-limiting examples illustrated in the accompanying drawings. Descriptions of well-known materials, fabrication tools, processing techniques, etc., are omitted so as not to unnecessarily obscure the invention in detail. It should be understood, however, that the detailed description and the specific examples, while indicating aspects of the invention, are given by way of illustration only, and are not by way of limitation. Various substitutions, modifications, additions, and/or arrangements, within the spirit and/or scope of the underlying inventive concepts will be apparent to those skilled in the art from this disclosure.

The terminology used herein is for the purpose of describing particular examples only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include (and any form of include, such as “includes” and “including”), and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more steps or elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable or suitable. For example, in some circumstances, an event or capacity can be expected, while in other circumstances the event or capacity cannot occur—this distinction is captured by the terms “may” and “may be.”

Several aspects of a biological sample identification system will now be presented with reference to various apparatus and methods. These apparatus and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in. one or more example embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media may include a random-access memory (RAM), a read-only memory (ROM), an electrically erasable programmable ROM (EEPROM), a flash memory, optical disk storage, magnetic disk storage, other magnetic storage devices, combinations of the aforementioned types of computer-readable media, or any other medium that can be used to store computer executable code in the form of instructions or data structures that can be accessed by a computer.

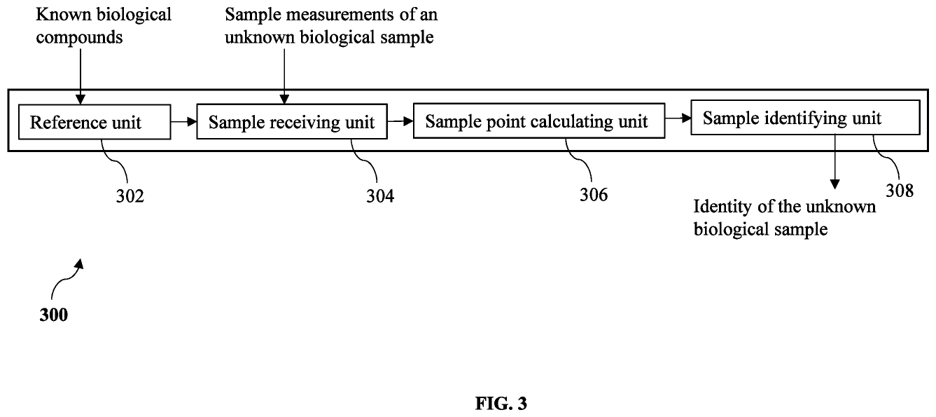

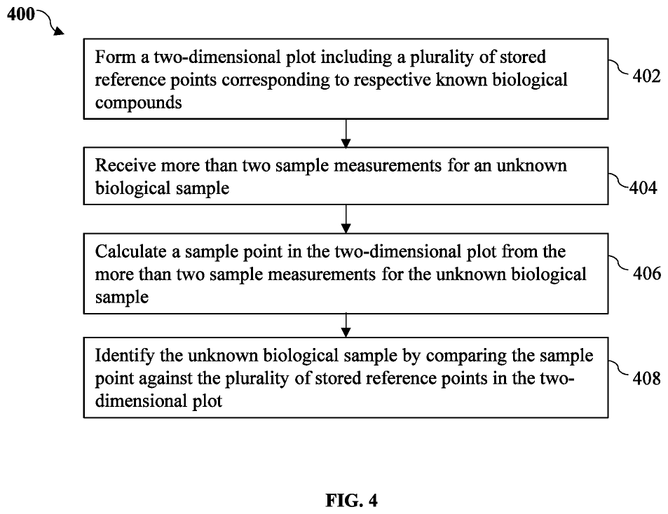

FIG. 3 shows a conceptual diagram of a system 300 for identifying an unknown biological sample according to various embodiments. The system 300 may include a reference unit 302, a sample receiving unit 304, a sample point calculating unit 306 and a sample identifying unit 308. FIG. 4 shows a flow diagram of a method 400 for identifying an unknown biological sample that may be implemented by the system 300 according to various embodiments. In various embodiments, the unknown biological sample may include one of the following: glycan, metabolite, antibody and any analyte. In various embodiments, the unknown biological sample may include one or more glycans such as but not limited to, glycosphingolipid glycan, N-glycan, O-glycan. In various embodiments, the unknown biological sample may include a procainamide-labelled glycan.

Referring to FIG. 4, according to various embodiments, at 402, the system 300 (e.g. the reference unit 302) may be configured to form a two-dimensional plot that may include a plurality of stored reference points corresponding to respective known biological compounds. By “known biological compounds”, it is meant that the structures or other characteristics of the biological compounds are known, but some known biological compounds may be theoretical compounds with structures or other characteristics theoretically predicted but not experimentally verified. At 404, the system 300 (e.g. the sample receiving unit 304) may be configured to receive more than two sample measurements for an unknown biological sample. At 406, the system 300 (e.g. the sample point calculating unit 306) may be configured to calculate a sample point in the two-dimensional plot from the more than two sample measurements for the unknown biological sample. At 408, the system 300 (e.g. the sample identifying unit 308) may be configured to identify the unknown biological sample by comparing the sample point against the plurality of stored reference points in the two-dimensional plot.

The method 400 will now be elaborated in greater detail.

### 402: Form a Two-Dimensional Plot Including a Plurality of Stored Reference Points Corresponding to Respective Known Biological Compounds

Referring to FIG. 4, at 402, the method 400 may include forming a two-dimensional plot including a plurality of stored reference points corresponding to respective known biological compounds.

In various embodiments, forming the two-dimensional plot at 402 may include calculating the reference points for the two-dimensional plot. Each reference point may be calculated from reference measurements, where each reference measurement may be for an attribute of the known biological compound the reference point corresponds to. The reference measurements may alternatively be referred to as training measurements/training datasets.

In various embodiments, an attribute may include one of the following: mass (m), mass to charge ratio (m/z), retention time, normalized retention time, glucose unit (GU), collisional cross section (CCS), tandem mass spectrometry (MSⁿ)/mass spectrometry (MS) fragmentation, measured shift in retention time after exoglycosidase treatment, measured shift in m/z after exoglycosidase treatment, measured shift in CCS after exoglycosidase treatment, measured shift in MSⁿ/MS fragmentation.

In various embodiments, each reference point may be calculated from a plurality of reference measurements for more than two attributes of the corresponding known biological compound, where each attribute may be different from another attribute.

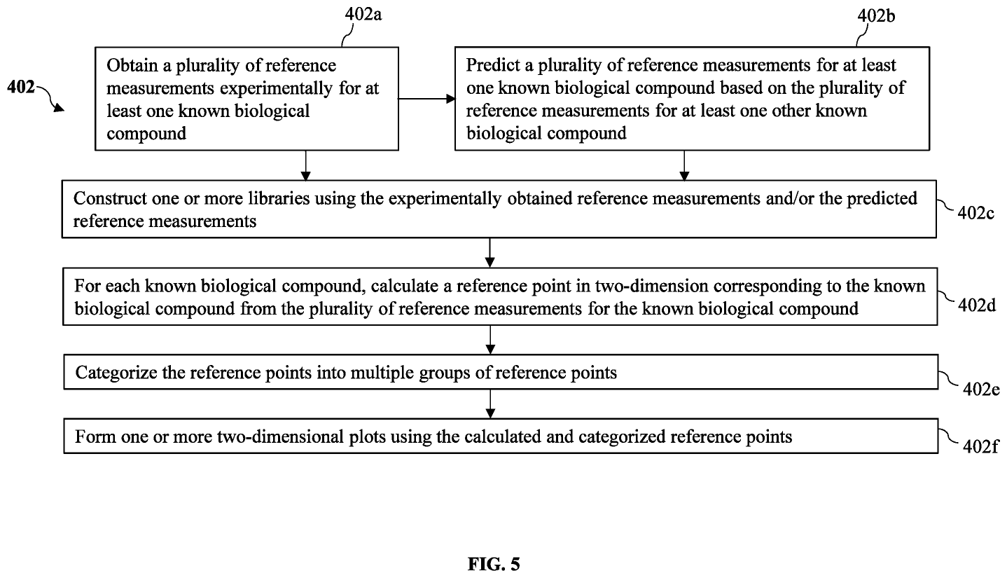

FIG. 5 shows a flow diagram of forming a two-dimensional plot at 402 in various embodiments. As shown in FIG. 5, each reference measurement may be obtained experimentally (e.g. at 402a) or by a machine learning algorithm (e.g. at 402b).

### 402a Obtain a Plurality of Reference Measurements Experimentally for at Least One Known Biological Compound

Referring to FIG. 5, at 402a, a plurality of reference measurements may be obtained experimentally for at least one known biological compound. For example, the known biological compound may be analysed with experimental devices to obtain the plurality of reference measurements. The experimental devices may include a complex combination of instruments/tools. For instance, the reference measurements for the known biological compound may be obtained by performing two or more of the following on the known biological compound: liquid chromatography, mass spectrometry, ion mobility, tandem mass spectrometry.

FIG. 6 shows a flow diagram of an example workflow that may be implemented at 402a to obtain a plurality of reference measurements experimentally for a known biological compound.

A sample 602 of the known biological compound may be provided for the workflow in FIG. 6. As shown in FIG. 6, at 604, exoglycosidase treatment may be performed on the sample 602 to obtain a cleaved sample 606 of the known biological compound. The exoglycosidase treatment of the sample 602 may involve exoglycosidase digest of the sample 602 by certain enzymes that cleave specific glycan monosaccharides. The exoglycosidase treatment of the sample 602 may provide additional structural information as the cleaving of the specific glycan monosaccharides can produce measurable shifts in various attributes.

Both the sample 602 and the cleaved sample 606 may be analysed with experimental devices including a LC device 608, a MS device 610, an IM device 612 and an MSⁿ device 614. Using these experimental devices 608-614, a LC data analysis may be performed on the sample 602 and the cleaved sample 606 at 616, a LC-MS data analysis may be performed on the sample 602 and the cleaved sample 606 at 618, a LC-MS-IM data analysis may be performed on the sample 602 and the cleaved sample 606 at 620 and a LC-IM-MS″ data analysis may be performed on the sample 602 and the cleaved sample 606 at 622. Using these data analyses 616-622, a plurality of reference measurements may be obtained for various attributes of the known biological compound.

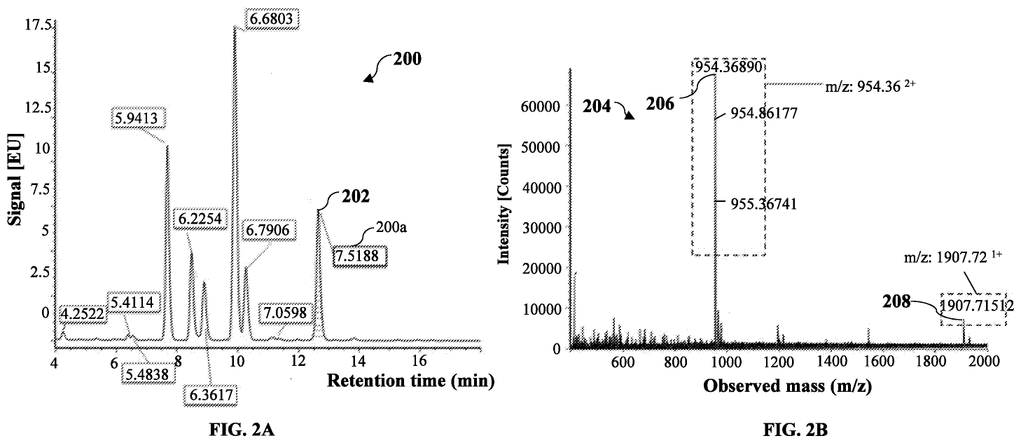

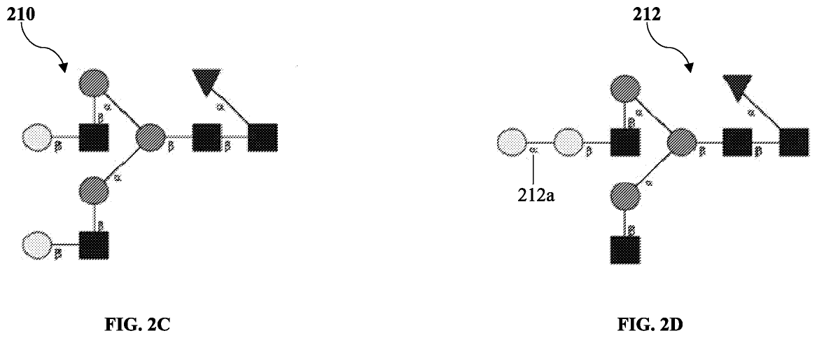

Examples of these attributes are shown in table 624. As shown in table 624, the attributes for the sample 602 may include GU (GU values may be obtained from the LC data analysis at 616), m/z of precursor ions (m/z precursor values may be obtained from the LC-MS data analysis at 618), CCS charge states 1, 2 and 3 (CCS values may be obtained from the LC-MS-IM data analysis at 620) and m/z of fragment ions (m/z fragment values may be obtained from the LC-IM-MSⁿ data analysis at 622). For example, if the sample 602 includes the isomer 210 in FIG. 2C, a reference measurement of 7.5719 may be obtained for the attribute “GU” and a reference measurement of 954.3667 may be obtained for the attribute “m/z precursor”. As shown in table 624, the attributes for the cleaved sample 606 may include measured shifts (A) in the above-mentioned attributes for the sample 602. The fragment ions may be diagnostic ions associated with characteristics of the samples 602, 606 and thus, may provide further structural information of the samples 602, 606. The CCS charge states 1, 2 and 3 may correspond respectively to the following three different charge states: singly charged ([M+H]¹⁺), doubly charged ([M+2H]²⁺), doubly charged, sodiated ([M+Na]²⁺ or [M+H+Na]¹⁺), and may alternatively be referred to as CCS[M+H]¹⁺, CCS[M+H]²⁺ and CCS[M+Na]²⁺.

### 402b Predict a Plurality of Reference Measurements for at Least One Known Biological Compound

Referring to FIG. 5, at 402b, a plurality of reference measurements may be predicted (instead of obtained experimentally) for at least one known biological compound. For example, the reference measurements may be predicted based on a plurality of reference measurements for at least one other known biological compound (which may be obtained experimentally from 402a).

In various embodiments, the reference measurements for a known biological compound may be obtained/predicted using a machine learning algorithm (or artificial intelligence (A.I.). As an example, the machine learning algorithm may be a regression model which may average the output of one or more of the following algorithms: multi-layer perceptron, random forest, and recursive neural network. The plurality of reference measurements may be predicted by the random forest, multi-layer perception and recursive neural network functions rf(X, ϕ_{rf}), mlp(x, ϕₘₗₚ) and rnn(x, ϕᵣₙₙ). In various embodiments, if the number of known biological compounds with experimentally obtained reference measurements is more than 10,000, functions rf(x, ϕ_{rf}), mlp(x, ϕₘₗₚ) and rnn(x, ϕᵣₙₙ) , may be optimized with deep learning algorithms where the number of parameters may be large (e.g. the number of variables in the ϕ's may be large). Otherwise, the number of parameters may be limited (e.g. the number of variables in the ϕ's may be restricted) as per the Vapnik-Chervonenkis (VC) dimension.

A more specific example of how reference measurements for a known biological compound may be predicted at 402b is elaborated below.

As described above, the machine learning algorithm may use the functions rf(x, ϕ_{rf}), mlp(x, ϕₘₗₚ) and rnn(x, ϕᵣₙₙ). Machine learning parameters ϕ_{rf}, ϕₘₗₚ, ϕᵣₙₙ of these functions may first be optimized using features of known biological compounds and reference measurements obtained experimentally for these known biological compounds. For example, a vector/array corresponding to each known biological compound may first be formed, where the vector/array may include scalar and categorical values describing features of the known biological compound. These vectors/arrays may then be inputted into the machine learning algorithm to obtain predicted measurements for the known biological compounds. The predicted measurements may then be compared against the experimentally obtained reference measurements of the known biological compounds. Based on the comparison, the machine learning parameters may be adjusted. This may continue until the predicted measurements are sufficiently close to the experimentally obtained reference measurements, or in other words, until the machine learning parameters are optimized. For example, the process may continue until an average difference between the predicted measurements and the experimentally obtained measurements is below a predetermined threshold.

To predict reference measurements for a known biological compound, a vector/array x corresponding to this known biological compound may first be formed, where this vector/array x may be similar to those inputted into the machine learning algorithm to optimize the machine learning parameters. In other words, the vector/array x may include scalar and categorical values describing features of the known biological compound. This vector/array x may be inputted into the machine learning algorithm, and the machine learning algorithm may use the functions rf(x, ϕ_{rf}), mlp(x, ϕₘₗₚ) and rnn(x, ϕᵣₙₙ) with the optimized machine learning parameters ϕ_{rf}, ϕₘₗₚ, ϕᵣₙₙ to predict reference measurements for the known biological compound. The reference measurements may be predicted based on the outputs of the functions rf(x, ϕ_{rf}), mlp(x, ϕₘₗₚ) and rnn(x, ϕᵣₙₙ) For example, the reference measurements may be predicted by taking averages of one or more of the outputs of the functions rf(x, ϕ_{rf}), mlp(x, ϕₘₗₚ) and rnn(x, ϕᵣₙₙ). Averaging may also be known as ensemble learning in machine learning and may help improve accuracy. The averaging method to predict the reference measurements may depend on how well each of the machine learning parameters ϕ's i.e. ϕ_{rf}, ϕₘₗₚ, ϕᵣₙₙ have been optimized. For instance, the reference measurements for the known biological compound may be predicted as [output of rf(x, ϕ_{rf})+output of rnn(x, ϕᵣₙₙ)]/2 when the parameters for the random forest algorithm and the recursive neural network model are optimized correctly but the parameter for the multi-layer perceptron algorithm is not.

402c Form One or More Libraries Using the Experimentally Obtained Reference Measurements and/or the Predicted Reference Measurements

Referring to FIG. 5, at 402c, one or more libraries may be constructed using the experimentally obtained reference measurements (from 402a) and/or the predicted reference measurements (from 402b).

In various embodiments, the experimentally obtained reference measurements may be used to construct an experimental multi-attribute library (or in short, experimental library); whereas the predicted reference measurements may be used to construct an in silico multi-attribute library (or in short, in silico library). Accordingly, the experimental library may include reference measurements for a set of known biological compounds, where for each known biological compound, the library may include experimentally obtained reference measurements for more than two attributes of the known biological compound. Similarly, the in silico library may include reference measurements for a set of known biological compounds, where for each known biological compound, the library may include predicted reference measurements for more than two attributes of the known biological compound.

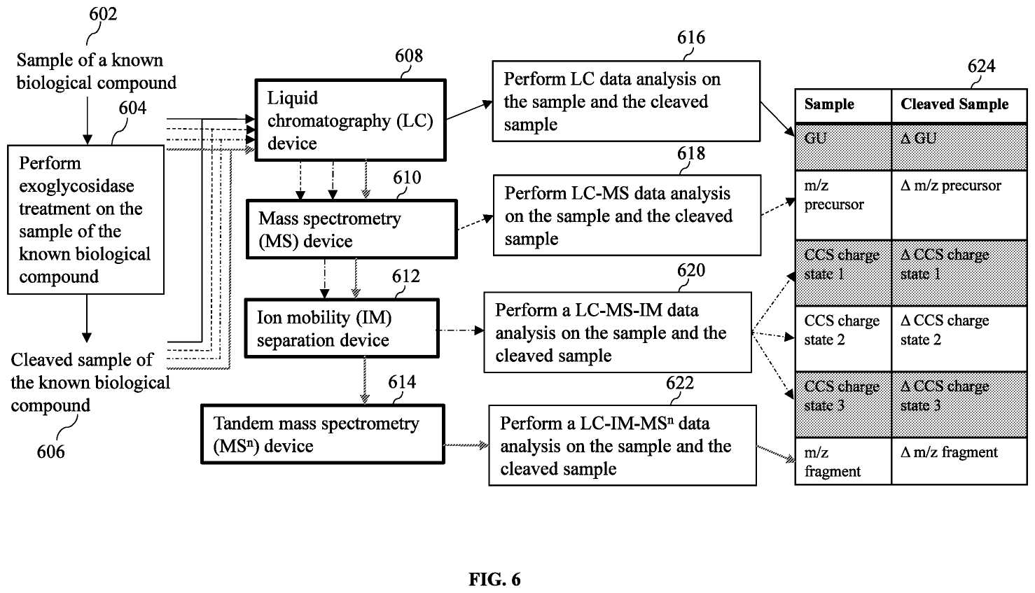

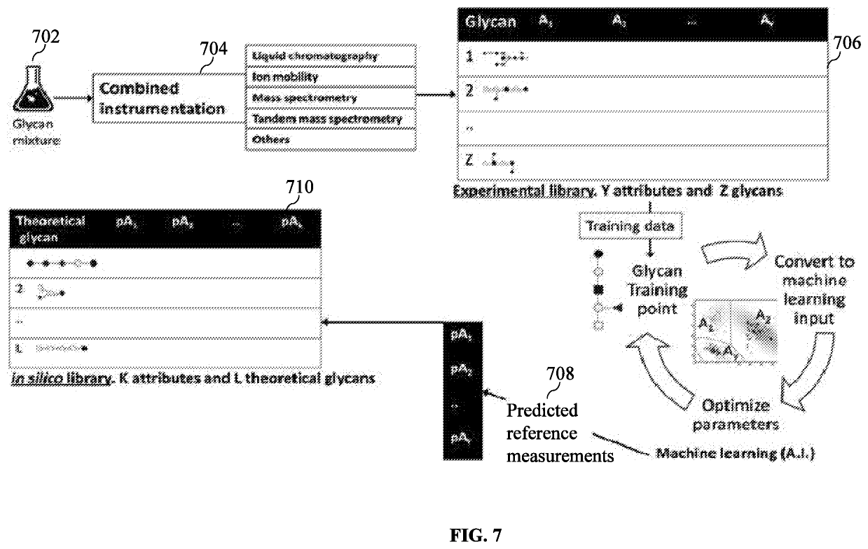

FIG. 7 shows an example workflow to form an experimental library and an in silico library for glycans. As shown in FIG. 7, a glycan mixture 702 including known biological compounds in the form of Z known glycans may be injected into a combined instrumentation system 704. The combined instrumentation system 704 may include various experimental devices such as but not limited to, LC, IM, MS, MSⁿ devices. Using the system 704, a total of Y reference measurements may be experimentally obtained for each known glycan, where each reference measurement may be for a respective one of Y attributes (A₁-A_{Y}) of the known glycan. The Y reference measurements for each of the Z known glycans may then be used to construct an experimental library in the form of a table 706. Referring to FIG. 7, the reference measurements in the experimental library may then be used to predict reference measurements 708 for K attributes (pA₁-pA_{K}) of each of L theoretically known glycans (or in short, L theoretical glycans). As shown in FIG. 7, the reference measurements in the experimental library may be used as training data to form a glycan training point which may then be converted into machine learning input for optimizing machine learning parameters of a machine learning algorithm. For example, the glycan training point may include features describing the Z known glycans, the machine learning input may include vectors/arrays including scalar and categorical values describing these features, and the machine learning parameters may be optimized using these vectors/arrays in the manner as described above. Alternatively, the machine learning input may include graphs (e.g. graphs including nodes (which may represent chemical elements (at a fine level) or monosaccharides (at a coarse level)), and edges/bonds connecting the nodes), where these graphs may describe the features of the Z known glycans and the machine learning parameters may be optimized using these graphs. K reference measurements for each of the L theoretical glycans may then be predicted with the machine learning algorithm using the optimized machine learning parameters and may then be used to construct an in silico library in the form of a table 710.

In some embodiments, two separate libraries, in particular, the experimental library and the in silico library may be constructed (for example, as shown in FIG. 7). However, in other embodiments, a single combined/dynamic library may be constructed using both the experimentally obtained reference measurements and the predicted reference measurements. In some other embodiments, 402b of method 400 may be omitted and only the experimental library may be constructed.

### 402d For Each Known Biological Compound, Calculate a Reference Point in Two-Dimension Corresponding to the Known Biological Compound

Referring to FIG. 5, after obtaining the reference measurements for known biological compounds at 402a and/or at 402b, a reference point in two-dimension corresponding to each known biological compound may be calculated at 402d. This reference point may be calculated from the plurality of reference measurements obtained (either experimentally at 402a or by prediction at 402b) for more than two attributes of the known biological compound. For example, a reference point may be calculated from the reference measurements obtained for the attributes “GU”, “CCS charge state 1” and “m/z precursor” of the known biological compound as shown in Table 624 of FIG. 6.

In various embodiments, each reference point may be calculated by performing principal component analysis on the plurality of reference measurements. Performing principal component analysis on the plurality of reference measurements may include transforming the plurality of reference measurements into a plurality of principal components. The principal components may be in an order such that variances of the principal components from a first principal component to a last principal component are in a descending order. Further, each principal component may be orthogonal to a next principal component in the order. A reference point may be formed in two-dimension using the first and second principal components. In one example, the reference point may be formed using the first and second principal components in this order, in other words, the first dimension of the reference point may include the first principal component and the second dimension of the reference point may include the second principal component. The first and second principal components usually cover a high variance (approximately greater than 0.75) so they may contain most of the information in the reference measurements.

An example of transforming a plurality of reference measurements (N reference measurements for each of k biological compounds) into a plurality of reference points is described below.
Let Rᵢ(x) denote the i^{th} reference measurement of the X^{th} biological compound for i=1, . . . , N and x=1, . . . , k.

$${\overline{R}}_{i} = {\sum }_{x = 1}^{k} \frac{{R}_{i} ( x )}{N} and {σ}_{i} = \frac{{\sum }_{x = 1}^{k} {( {R}_{i} ( x ) - {\overline{R}}_{i} )}^{2}}{N - 1}$$

which are respectively the mean and standard deviation values of the i^{th} reference measurement over all k biological compounds may then be calculated.
Let \hat{R}ᵢ(x) denote the i^{th} reference measurement of the x^{th} biological compound standardized to mean 0 and standard deviation 1 using the following equation:

$${\hat{R}}_{i} ( x ) = \frac{{R}_{i} ( x ) - {\overline{R}}_{i}}{{σ}_{i}} .$$

The values \hat{R}ᵢ(x) may be calculated for all reference measurements i=1, . . . , N.
The x^{th} biological compound and its N reference measurements may then be mapped to a reference point (P₁, P₂) in two-dimensional, where P₁ is the first principal component defined as the linear combination: P₁=α₁₁\hat{R}₁(x)+α₂₁\hat{R}₂(x)+ . . . +α_{N1}\hat{R}_{N}(x) and P₂ is the second principal component defined as the linear combination: P₂=α₁₂\hat{R}₁(x)+α₂₂\hat{R}₂(x)+ . . . +α_{N2}\hat{R}_{N}(x). The coefficients αₓ₁ for x=1, . . . , N are real numbered scalar values for the first principal component and the coefficients αₓ₂ for x=1, . . . N are real numbered scalar values for the second principal component.
The coefficients α₁₁, α₂₁, . . . , α_{N1} used to compute the first principal component and the scalar values α₁₂, α₂₂, . . . , α_{N2} used to compute the second principal component may be calculated as follows:
   the covariance between i^{th} and j^{th} reference measurements Rᵢ and Rⱼ of the x^{th} biological compound,

$$cov ( {R}_{i} , {R}_{j} ) = \frac{{\sum }_{x = 1}^{k} ( {R}_{i} ( x ) - {\overline{R}}_{i} ) ( {R}_{j} ( x ) - {\overline{R}}_{j} )}{N - 1}$$

may first be used to construct a covariance matrix C. The covariance matrix, C, contains all possible covariance's between all N reference measurements:

$$C = ( \begin{matrix}cov ( {R}_{1} , {R}_{1} ) & cov ( {R}_{1} , {R}_{2} ) & … & cov ( {R}_{1} , {R}_{N} ) \\ cov ( {R}_{2} , {R}_{1} ) & cov ( {R}_{2} , {R}_{2} ) & … & cov ( {R}_{2} , {R}_{N} ) \\ ⋮ & ⋮ & ⋱ & ⋮ \\ cov ( {R}_{N} , {R}_{1} ) & cov ( {R}_{N} , {R}_{2} ) & … & cov ( {R}_{N} , {R}_{N} )\end{matrix} )$$

Solving the equation C. v=λ. v for v and λ, the eigenvectors v's which are usually in the form of N×1 non-zero vectors and the eigenvalues λ's which are usually in the form of scalar values may be determined. For N reference measurements, there are N eigenvectors v₁, . . . , v_{N}, and N corresponding eigenvalues λ₁, . . . , λ_{N}. The first two principal eigenvectors v₁=(α₁₁, α₂₁, . . . , α_{N1}) and v₂=(α₁₂, . . . , α₂₂, . . . , α_{N2}) are the ones corresponding to the two largest eigenvalues λ₁ and λ₂; and the scalar values (α₁₁, α₂₁ , . . . , α_{N1}) and (α₁₂, α₂₂, . . . , α_{N2}) from the first two principal eigenvectors v₁ and v₂ may be used to compute the principal components P₁ and P₂ for any N reference measurements; P₁ and P₂ are orthogonal; and the variance covered by and P₂ is

$$\frac{{λ}_{1} + {λ}_{2}}{{\sum }_{i = 1}^{N} {λ}_{i}} .$$

Using the above-described method, k reference points in two-dimension corresponding to the k biological compounds may be calculated, with each reference point defined by (P₁, P₂) calculated in the manner described above. These reference points (P₁, P₂) may subsequently be used to form a two-dimensional plot to visualize the biological compounds in a simple manner on for example, a device (e.g. computer monitor or hand-held device).

In some embodiments, the reference points may be calculated from reference measurements using algorithms other than principal component analysis. Any algorithm known to one skilled in the art may be used as long as the algorithm is capable of compressing the plurality of reference measurements of known biological compounds into two-dimensional reference points without significantly diminishing the accuracy of identifying an unknown biological sample using these reference points. For example, the vectors v₁=(α₁₁, α₂₁, . . . , α_{N1}) and v₂=(α₁₂, α₂₂, α_{N2}) need not be eigenvectors of the covariance matrix C above and other methods may be employed to calculate the vectors v₁=(α₁₁, α₂₁, . . . , α_{N1}) and v₂=(α₁₂, α₂₂, . . . , α_{N2}) used for calculating the reference point (P₁, P₂). These methods may include neural networks and variants thereof such as auto-encoders, denoising auto-encoders and ladder networks. Other algorithms capable of calculating a two-dimensional point (P₁, P₂) from the reference measurements, where P₁ and P₂ may not necessarily be principal components, may also be used. These may include neural networks and variants thereof such as auto-encoders, denoising auto-encoders and ladder networks.

402e Categorize the Reference Points into Multiple Groups of Reference Points

Referring to FIG. 5, forming the two-dimensional plot may further include categorizing, at 402e, the reference points into multiple groups of reference points.

In various embodiments, the known biological compounds (for which the reference points are calculated at 402c) may be categorized into multiple groups of isomers, and the reference points may be categorized into multiple groups of reference points corresponding to respective groups of isomers. Each reference point may be categorized into the group of reference points corresponding to the group of isomers into which the corresponding known biological compound is categorized.

### 402f Form One or More Two-Dimensional Plots Using the Calculated and Categorized Reference Points

Referring to FIG. 5, forming the two-dimensional plot may further include forming the plot using the calculated and categorized reference points.

In some embodiments, a single two-dimensional plot may be formed using the reference points calculated from both the experimentally obtained reference measurements and the predicted reference measurements. In other words, this single two-dimensional plot may be a compressed space of a combined library including both the experimental library and the in silico library.

In alternative embodiments, two separate two-dimensional plots may be formed, with one formed using the reference points calculated from the experimentally obtained reference measurements and the other formed using the predicted reference measurements. In other words, these two plots may respectively be a compressed space of the experimental library and a compressed space of the in silico library.

In various embodiments, each two-dimensional plot may be referred to as a MAGSpace.

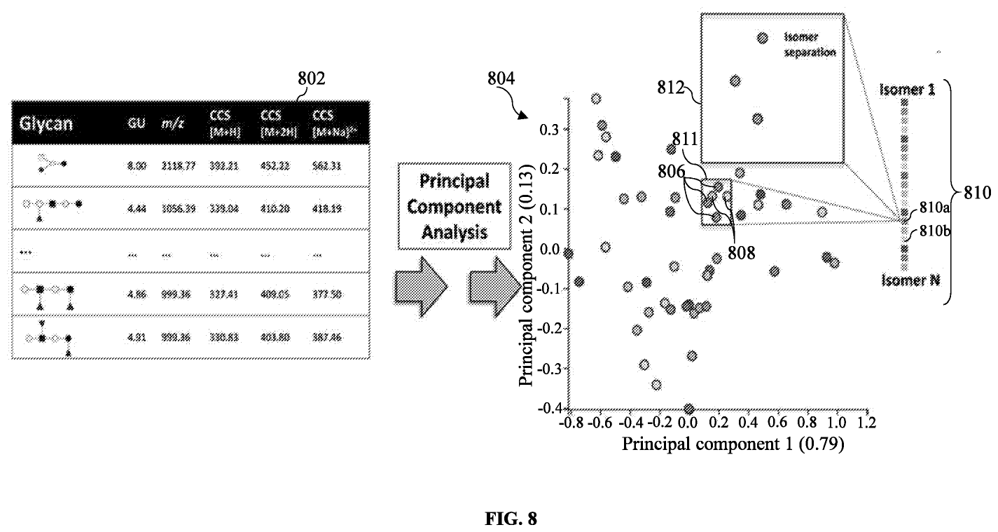

FIG. 8 shows an example workflow to form a two-dimensional plot using the calculated and categorized reference points. As shown in FIG. 8, the reference points may be calculated by performing principal component analysis on a plurality of reference measurements in a library 802. These reference measurements may be for a plurality of glycans, such as but not limited to, GSL glycans. First and second principal components (Principal component 1 and Principal component 2) may be obtained from the principal component analysis. Two-dimensional reference points may be defined by these principal components and may be used to construct a two-dimensional plot 804 (where the second principal components of the reference points are plotted against the first principal components). Accordingly, the plot 804 may include a plurality of reference points e.g. reference points 806, 808 in two-dimension.

As shown in FIG. 8, the known biological compounds may be categorized into multiple groups of isomers 810 and the reference points 806, 808 may be categorized into multiple groups of reference points corresponding to respective groups of isomers 810a, 810b. For example, for the reference points 806, 808 in the box 811, the reference points 806 may correspond to known biological compounds belonging to a first group of isomers 810a and the reference points 808 may correspond to known biological compounds belonging to a second group of isomers 810b. Thus, the reference points 806 may be categorized into the same group 812 of reference points, whereas the reference points 808 may be categorized into another group of reference points (not shown in FIG. 8). The known biological compounds may be categorized into the multiple groups of isomers based on various characteristics of these compounds. In one example, each known biological compound may be categorized based on a mass value of the known biological compound. In various embodiments, the two-dimensional plot may be formed by differentiating the reference points in different groups using different shades (as shown in FIG. 8) or other characteristics such as colors or sizes of the points, so as to facilitate visualization of the different groups of reference points.

### Updating the Libraries and Two-Dimensional Plot(s)

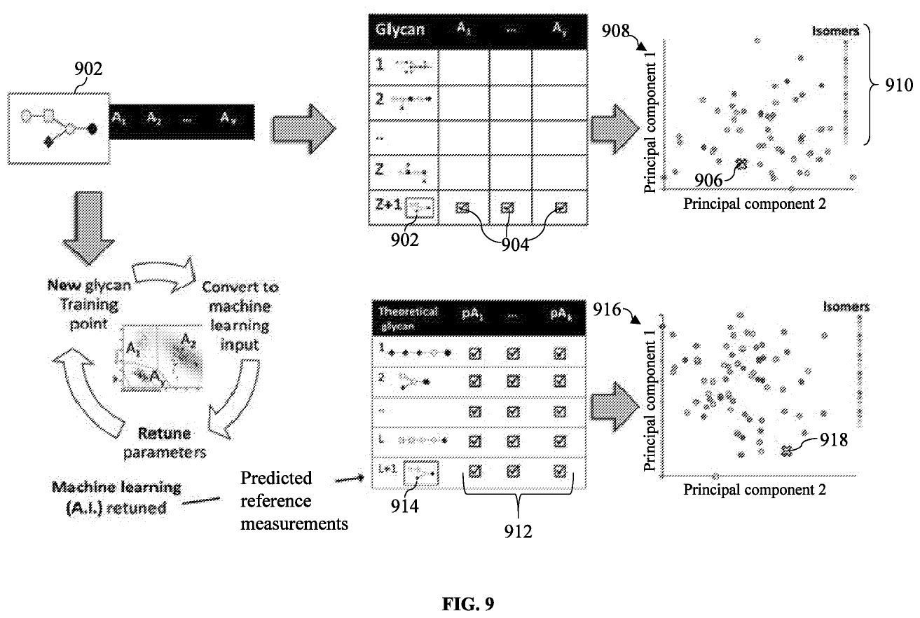

In various embodiments, the experimental library and the in silico library may be updated when a new known biological compound with experimentally determined reference measurements is available. FIG. 9 shows an example workflow of updating the experimental library and the in silico library of FIG. 7.

As shown in FIG. 9, a total of Y reference measurements 904 may be obtained for a new glycan 902, where each reference measurement may be for a respective one of Y attributes (A₁-A_{Y}) of the new glycan 902. A reference point 906 in two-dimension corresponding to the new glycan 902 (glycan Z+1) may then be calculated in a similar manner as described above with reference to 402d of FIG. 5, and may be added to a two-dimensional plot 908 formed previously with the experimentally determined reference measurements of glycans 1 to Z. The new glycan 902 may be categorized into one of the plurality of groups 910 of isomers and the reference point 906 may be categorized into a group of reference points corresponding to the group of isomers the new glycan is categorized into.

The reference measurements of the new glycan 902 (glycan Z+1) may also be input to the machine learning algorithm previously optimized by the reference measurements of the glycans 1 to Z. With the reference measurements of all the glycans 1 to Z+1, a new glycan training point may be formed and converted into machine learning input to retune (or in other words, re-optimize) the parameters of the machine learning algorithm. Reference measurements 912 may then be predicted for all the L theoretical glycans previously present in the in silico library and for a new theoretical glycan 914 (glycan L+1) further included in the in silico library. The new theoretical glycan 914 (glycan L+1) may be similar to the new glycan 902. A two-dimension plot 916 may be formed, where the plot 916 may include reference points calculated from the newly predicted reference measurements 912 in a similar manner as described above with reference to 402d of FIG. 5. For example, the reference point 918 may correspond to the theoretical glycan L+1 and may be calculated using the reference measurements predicted for this theoretical glycan L+1. Note that although FIG. 9 shows two separate plots 908, 916, with the plot 908 formed with experimentally determined reference measurements and the plot 916 formed with predicted reference measurements, only a single two-dimensional plot may be formed with both experimentally determined reference measurements and predicted reference measurements in some alternative embodiments.

In some embodiments, formation of two-dimensional plot(s) at 402 may be performed only once or the two-dimensional plot(s) may be updated at 402 only whenever reference measurements for a new known biological compound are available. On the other hand, 404-406 may be repeatedly performed to identify different unknown biological compounds using the same two-dimensional plot(s). In some embodiments, 402 may be totally omitted and one or more two-dimensional plots, each having a plurality of reference points corresponding to respective known biological compounds similar to those formed in the manner as described above, may be provided for performing 404-406 of method 400.

### 404 Receive More Than Two Sample Measurements for an Unknown Biological Sample

Referring to FIG. 4, the method 400 may further include receiving more than two sample measurements for an unknown biological sample at 404. Each sample measurement may be for an attribute of the unknown biological sample. In various embodiments, a plurality of sample measurements may be obtained experimentally for respective attributes of the unknown biological sample in a manner similar to that described with reference to 402a of FIG. 5.

406 Calculate a Sample Point in the Two-Dimensional Plot from the More Than Two Sample Measurements for the Unknown Biological Sample

Referring to FIG. 4, the method 400 may include calculating a sample point in the two-dimensional plot (formed at 402) from the more than two sample measurements for the unknown biological sample received at 406. In other words, the unknown biological sample may be mapped to the two-dimensional plot. This mapping may be referred to as MAGMap.

In various embodiments, calculating the sample point may include performing principal component analysis on the more than two sample measurements.

As previously described, transforming the plurality of reference measurements into a plurality of principal components may include calculating a plurality of principal component parameters such as R̅ᵢ, σᵢ and eigenvectors v₁=(α₁₁, α₂₁, . . . , α_{N1}) and v₂=(α₁₂, α₂₂, . . . , α_{N2}). Performing principal component analysis on the more than two sample measurements may include using this plurality of principal component parameters in a similar manner as that described above for calculating reference points using the principal component parameters. For example, this may include transforming the plurality of sample measurements into a plurality of principal components using the principal component parameters derived from the reference measurements, where the principal components from the sample measurements may be in an order such that variances of the principal components from a first principal component to a last principal component are in a descending order and each principal component may be orthogonal to a next principal component in the order. The first and second principal components from the principal component analysis of the sample measurements may then be used to form the sample point in the two-dimensional plot. The first and second principal components may be orthogonal to each other.

A more specific example of transforming a plurality of sample measurements (N sample measurements for an unknown biological compound) into a plurality of principal components to form a sample point is described below.
For each sample measurement Sᵢ, where i=1, . . . , N, a standardized/normalized sample measurement may first be calculated using the equation Ŝᵢ=(Sᵢ−R̅ᵢ)/σᵢ where R̅ᵢ and σᵢ are the principal component parameters, in particular the mean and standard deviation values of the i^{th} reference measurement over all k biological compounds respectively. These may be calculated from the reference measurements in the manner as described above.
The first principal component P₁ may then be calculated as the linear combination: P₁=(α₁₁Ŝ₁+α₂₁Ŝ₂+ . . . +α_{N1}Ŝ_{N} and the second principal component P₂ may be calculated as the linear combination: P₂=α₁₂Ŝ₁+α₂₂Ŝ₂+ . . . +α_{N2}Ŝ_{N} where αₓ₁ and αₓ₂ for x=1, . . . N may be derived from the reference measurements as described above.

In this example, the number of sample measurements (each sample measurement for one attribute) may be equal to the number of reference measurements (each reference measurement for one attribute), and the attributes the sample measurements are for may correspond to the attributes the reference measurements are for. This allows the transformation of the sample measurements into the principal components using the principal component parameters obtained with the reference measurements.

Further, in this example, the sample measurements may be mapped to the sample point (P₁, P₂) in two-dimension and the sample point (P₁, P₂) may be placed in the two-dimensional plot formed in 402. This may allow one to use the two-dimensional plot to visualize where the sample point is situated relative to the reference points in a clear manner (as compared to using a representation with more than two dimensions). The visualization may be performed on for example, a device (e.g. computer monitor or hand-held device). The reference points near the sample point correspond to known biological compounds similar to the unknown biological compound. Knowledge of such similar known biological compounds may be useful.

408 Identify the Unknown Biological Sample by Comparing the Sample Point Against the Plurality of Reference Points in the Two-Dimensional Plot

Referring to FIG. 4, the method 400 may include identifying, at 408, the unknown biological sample by comparing the sample point against the plurality of reference points in the two-dimensional plot (formed at 402). In various embodiments, identifying the unknown biological sample at 408 may include determining a reference point nearest to the sample point in the two-dimensional plot (e.g. determining a nearest reference point where a Euclidean distance between the nearest reference point and the sample point is smaller as compared to Euclidean distances between the remaining reference points and the sample point) and identifying the unknown biological sample as the known biological compound corresponding to the determined nearest reference point.

In a first example, only a single two-dimensional plot with reference points calculated from both experimentally obtained reference measurements and predicted reference measurements may be formed at 402, and the sample point may be compared against all the reference points in this two-dimensional plot. For example, all the reference points may first be categorized into different groups of reference points corresponding to respective groups of isomers. Each reference point may be categorized based on the group of isomer the corresponding known biological compound belongs to. Prior to determining the reference point nearest to the sample point in the two-dimensional plot, the unknown biological sample may be categorized into one of the multiple groups of isomers (based on for example, its m/z value) and only the reference points in the group corresponding to this group of isomers may be retained. The nearest reference point may then be selected/determined from these retained reference points.

In a second example, separate two-dimensional plots, one from experimentally obtained reference measurements and the other from predicted reference measurements, may be formed at 402. The reference points calculated from experimentally obtained reference measurements may be categorized into a first set of groups of reference points corresponding to respective first groups of isomers, and the reference points calculated from predicted reference measurements may be categorized into a second set of groups of reference points corresponding to respective second groups of isomers.

In this example, a first attempt to identify the unknown biological sample may be made using the plot from the experimentally determined reference measurements, and if the unknown biological sample is not found in this plot, a second attempt to identify the unknown biological sample may then be made using the plot from the predicted reference measurements. For each plot, the attempt to identify the unknown biological sample may include categorizing the unknown biological sample into one of the multiple groups of isomers corresponding to the respective groups of reference points in that plot, and retaining only the reference points in the group corresponding to the group of isomers the unknown biological sample is categorized into. The nearest reference point may then be selected/determined from these retained reference points.

For the first attempt, a sample point calculated in two-dimension (using for example, principal component analysis) may be compared against the reference points to determine a nearest reference point. However, prior to determining the nearest reference point, the unknown biological sample may be categorized into one of the multiple first groups of isomers and only the reference points in the group corresponding to this group of isomers may be retained. The nearest reference point may then be selected/determined from the retained reference points.

If the unknown biological sample does not belong to any one of the first groups of isomers corresponding to the first set of groups of reference points, the second attempt may be carried out by comparing the sample point against the reference points calculated from predicted reference measurements. Similarly, the unknown biological sample may be categorized into one of the multiple second groups of isomers corresponding to the second set of groups of reference points, and only the reference points in the group (in the second set) corresponding to the group of isomers into which the unknown biological sample is categorized may be retained. The nearest reference point may then be determined from these retained reference points. Since the reference points calculated via machine learning are part of an in silico library which includes almost all possible combinations of biological compounds, there is a low chance of failing to find a group of reference points which correspond to the group of isomers into which the unknown biological compound is categorized.

In some examples, a single two-dimensional plot may be formed at 402 from both experimentally obtained reference measurements and predicted reference measurements, and first and second attempts similar to those described above in the second example may still be made. In other words, the reference points in this single two-dimensional plot may be separated into first and second sets of groups of reference points and the attempts may be made accordingly as described above.

In various embodiments, identifying the unknown biological sample may further include calculating a distance between the sample point and the determined nearest reference point, and calculating an accuracy score based on this distance. In other words, a distance-based scoring approach may be used. In various embodiments, a mathematical distance (for example, a Euclidean distance between the sample point and the determined nearest reference point) may be used to characterize the unknown biological sample. In various embodiments, the accuracy score may be the distance between the sample point and the determined nearest reference point. In various embodiments, the accuracy score may include one of the following: a low confidence score, a medium confidence score, a high confidence score.

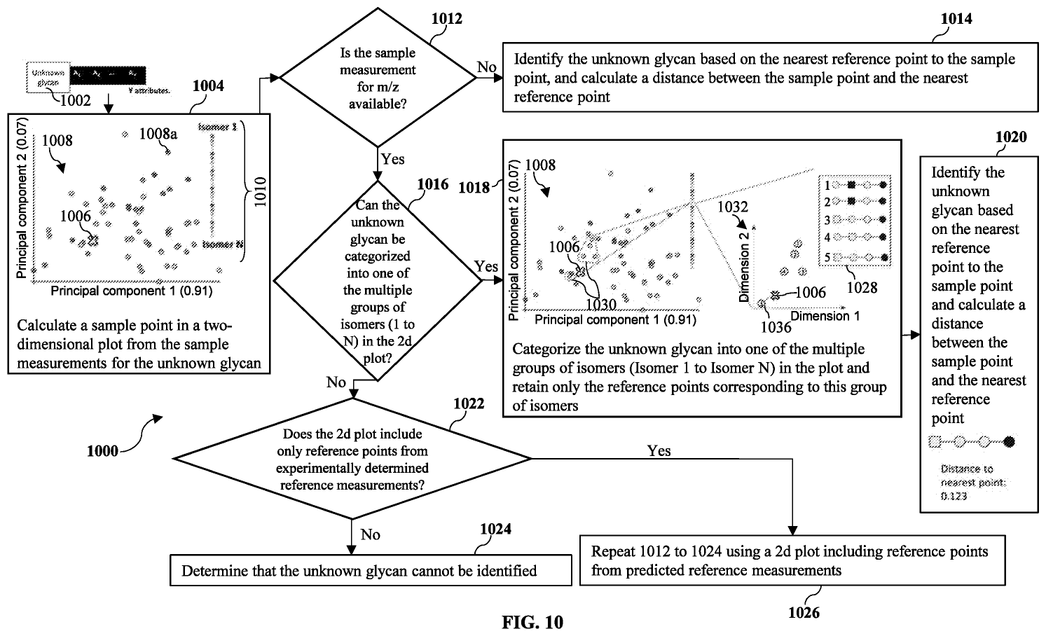

FIG. 10 shows an example workflow 1000 of calculating a sample point in two-dimension for an unknown biological sample and identifying the unknown biological sample. As shown in FIG. 10, the unknown biological sample may be in the form of an unknown glycan 1002 and a plurality of Y sample measurements may be obtained for respective ones of Y attributes (A₁ to A_{Y}) of the unknown glycan 1002. The workflow 1000 may include 1004 which may correspond to 406 of method 400. At 1004, the workflow 1000 may include mapping the unknown biological sample into a two-dimensional plot 1008. For example, a sample point 1006 may be calculated in two-dimension and added into the two-dimensional plot 1008. As shown in FIG. 10, the two-dimensional plot 1008 may include a plurality of reference points (e.g. reference point 1008a) categorized into multiple groups of reference points corresponding to respective groups 1010 of isomers. In FIG. 10, each group of reference points is shown in a different shade from the other groups of reference points.

The workflow 1000 may further include 1012 to 1026 which may correspond to 408 of method 400.

At 1012, it may be determined whether the sample measurement for m/z of the unknown glycan is available and if not, at 1014, the unknown glycan may be identified based on the nearest reference point to the sample point in the two-dimensional plot (e.g. the unknown glycan may be identified as the known glycan corresponding to the nearest reference point) and a distance between the sample point and the nearest reference point may be calculated. If the sample measurement for m/z of the unknown glycan is available, at 1016, it may be determined if the unknown glycan can be categorized into one of the multiple groups 1010 of isomers using the sample measurement for m/z of the unknown glycan. If yes, the unknown glycan may be categorized into the group 1028 of isomers which corresponds to the group 1030 of reference points. Only the reference points in this group 1030 may be retained as shown by plot 1032 (which may be referred to as a reduced MAGSpace). The reference point 1036 in this group 1030 nearest to the sample point 1006 may then be determined and at 1020, the unknown glycan may be identified as the glycan corresponding to this nearest reference point 1036. Further, at 1020, a distance between the sample point 1006 and the determined nearest reference point 1036 may be calculated as 0.123, and an accuracy score may subsequently be determined based on this distance.

If at 1016, it is determined that the unknown glycan cannot be categorized into one of the multiple groups 1010 of isomers, it is determined at 1022 whether the two-dimensional plot includes only reference points from experimentally determined reference measurements. If not, then at 1024, it may be determined that the unknown glycan cannot be identified. If yes, then at 1026, 1012 to 1024 may be repeated using a two-dimensional plot including reference points from predicted reference measurements.

Form and use Multiple Two-Dimensional Plots from Different Numbers of Attributes

In various embodiments, the method 400 for identifying an unknown biological sample may include using multiple two-dimensional plots, where each plot may be formed from a different number of attributes as compared to another plot.

Occasionally, there may be a failure in obtaining sample measurements for one or more attributes for an unknown biological sample. This may be due to the instrument used in obtaining the attribute. For example, varying signal intensities of the unknown biological sample (or analyte) in a MS instrumentation may result in a lack of sample measurements for some attributes for the unknown biological sample. If the match between the sample measurements and the reference measurements is poor, a fault may arise. For example, if sample measurements are obtained for three attributes for an unknown biological sample, but a two-dimensional plot formed from four attributes of known biological compounds is used to identify the unknown biological sample, a poor match may occur and the accuracy of identifying the unknown biological sample may be affected.

To alleviate the above problem, the experimental library, in silico library, or combined library may be dynamically divided into permutations of attributes to account for the missing attributes. This may be done by using multiple two-dimensional plots formed from different numbers of attributes. This can allow a better match between sample measurements and the reference measurements (in terms of the number of measurements and the attributes the measurements are for).

The number of two-dimensional plots in each library (experimental, in silico or combined) may be dependent on the total number of attributes with reference measurements for the known biological compounds available. For example, if there are y attributes with reference measurements available, then a total of

$$N = 1 + {\sum }_{k = 1}^{y - 1} \frac{y !}{( y - k ) ! k !}$$

MAGSpaces) may be used to identify an unknown biological sample.

In a more specific example, if there are four attributes including attributes A, B, C and D with reference measurements available, a total of 15 plots may be used to improve the accuracy of identifying the unknown biological sample. These plots may include:
(i) 1 two-dimensional plot formed from all four attributes (A, B, C, D)
(ii) 1 two-dimensional plot formed from three attributes (A, B, C)
(iii) 1 two-dimensional plot formed from three attributes (A, B, D)
(iv) 1 two-dimensional plot formed from three attributes (A, C, D)
(v) 1 two-dimensional plot formed from three attributes (B, C, D)
(vi) 6 two-dimensional plots, each formed from two attributes (A, B), (A, C), (A, D), (B, C), (B, D) or (C, D)
(vii) 4 plots, each formed from a single attribute A, B, C or D

Principal component analysis may be used to calculate the reference points for the plots formed from more than two attributes but may not be needed to calculate the reference points for the plots formed from one or two attributes. In other words, principal component analysis may be used to calculate the reference points for the plots stated in (i)-(v) above, whereas principal component analysis may not be needed to calculate the reference points for the plots stated in (vi)-(vii) above.

In this example, if sample measurements are obtained for only three attributes (A,C,D) for an unknown biological sample, then only the following plots out of the above plots (i)-(vii) may be used:

1 two-dimensional plot formed from three attributes (A,C,D)

1 two-dimensional plot formed from two attributes (A,C)

1 two-dimensional plot formed from two attributes (A,D)

1 two-dimensional plot formed from two attributes (C,D)

1 plot formed from a single attribute A

1 plot formed from a single attribute C

1 plot formed from a single attribute D

In other words, in this example, the method 400 may include using a first two-dimensional plot formed from three attributes (A, C, D), at least one further two-dimensional plot formed from two attributes (A, C or A, D or C, D), and at least one further two-dimensional plot formed from a single attribute (A or C or D). Each two-dimensional plot may include a plurality of stored reference points corresponding to respective known biological compounds. Each reference point of the first two-dimensional plot may be calculated from three reference measurements for the three attributes (A, C, D) of the corresponding known biological compound. A second two-dimensional plot may be one of the further plots formed from two attributes, and each reference point of the second two-dimensional plot may be calculated from two reference measurements for two attributes (A, C or A, D or C, D) of the corresponding known biological compound. A third two-dimensional plot may be one of the further plots formed from a single attribute, and each reference point of the third two-dimensional plot may be calculated from one reference measurement for the single attribute (A or C or D) of the corresponding known biological compound.

The three sample measurements for the three attributes (A, C, D) of the unknown biological sample may then be mapped to the two-dimensional plots. For example, a first sample point in the first two-dimensional plot, a second sample point in the second two-dimensional plot and a third sample point in the third two-dimensional plot may be calculated based on three sample measurements, two sample measurements and one sample measurement respectively for the unknown biological sample.

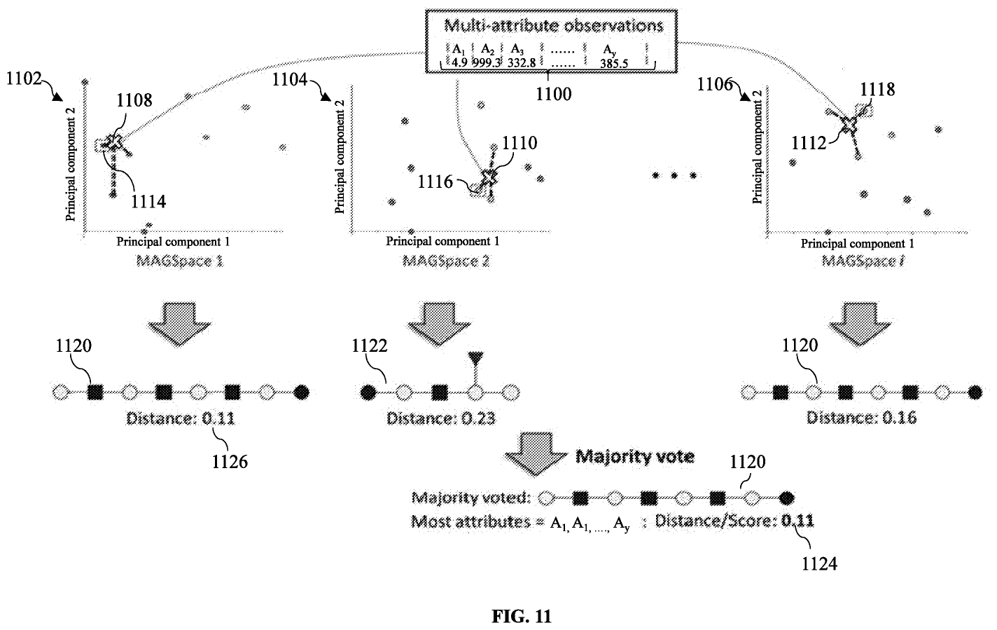

FIG. 11 shows an example workflow for the method 400 that may include forming and using a plurality of two-dimensional plots.

Referring to FIG. 11, a first two-dimensional plot 1102 (MAGSpace 1) may be formed (at 402) from a first number (Y) of attributes. In particular, each reference point of the first two-dimensional plot 1102 may be calculated from a first number (Y) of reference measurements 1100 for the first number (Y) of attributes of the corresponding known biological compound.

The method 400 may include forming/generating (at 402) further two-dimensional plots 1104, 1106 (e.g. MAGSpace 2, MAGSpace i in FIG. 11). Each further plot 1104, 1106 may include a plurality of stored reference points corresponding to respective known biological compounds. Each reference point of the further plot 1104, 1106 may be calculated from at least one reference measurement for at least one attribute of the corresponding known biological compound. In various embodiments, for each further plot 1104, 1106, the number of attributes from which the reference points are calculated may differ from the first number (Y) and may also differ from the number of attributes from which the reference points in a different further plot 1104, 1106 are calculated. In other words, each further two-dimensional plot 1104, 1106 may be formed from a different number of attributes as compared to another two-dimensional plot 1102, 1104, 1106. In some embodiments, the number of attributes from which the reference points are calculated for each further plot 1104, 1106 may be smaller than the first number (Y). For example, referring to FIG. 11, the first two-dimensional plot 1102 may be formed from Y attributes, and the further two-dimensional plots 1104, 1106 may be formed from Y-1 and 2 attributes respectively.

In various embodiments, the method 400 may include using the first two-dimensional plot 1102 and each of the further plots 1104, 1106.

For instance, the method 400 may include calculating (at 406) a sample point in the first two-dimensional plot 1102 from the sample measurements for the unknown biological sample. For example, the number of sample measurements for the unknown biological sample received at 404 may be equal to the first number (Y) and a sample point in the first two-dimensional plot 1102 may be calculated using these sample measurements. For example, referring to FIG. 11, a sample point 1108 may be calculated in the first two-dimensional plot 1102. The sample point 1108 may be calculated using sample measurements for the Y attributes used to form the first two-dimensional plot 1102.

The method 400 may also include calculating a sample point in each of the plurality of further two-dimensional plots 1104, 1106 based on at least one sample measurement for the unknown biological sample. For example, the sample point 1110 in the further plot 1104 may be calculated using Y-1 sample measurements for the Y-1 attributes used to form the further plot 1104, and the sample point 1112 in the further plot 1106 may be calculated using sample measurements for the two attributes used to form the further plot 1106.

In various embodiments, the method 400 may further include for each two-dimensional plot 1102, 1104, 1106, determining a reference point nearest to the sample point 1108, 1110, 1112 in the two-dimensional plot 1102, 1104, 1106. For example, referring to FIG. 11, a reference point 1114 nearest to the sample point 1108 in the first two-dimensional plot 1102, a reference point 1116 nearest to the sample point 1110 in the further two-dimensional plot 1104 and a reference point 1118 nearest to the sample point 1112 in the further two-dimensional plot 1106 may be determined. In some embodiments, a workflow similar to the workflow 1000 of FIG. 10 may instead be performed for each two-dimensional plot 1102, 1104, 1106 to identify a nearest reference point in each of these plots 1102, 1104, 1106.

The method 400 may also include identifying the unknown biological sample as the known biological compound corresponding to the most number of determined nearest reference points. As shown in FIG. 11, the reference point 1114 and the reference point 1118 may correspond to a first known glycan 1120 whereas the reference point 1116 may correspond to a second known glycan 1122. In this example, based on a majority vote, the unknown glycan may be identified as the first known glycan 1120 since this first known glycan 1120 corresponds to two out of three of the determined nearest reference points 1114, 1116, 1118. In other words, the first known glycan 1120 may be the majority voted glycan that has appeared the most frequently in all the MAGSpaces.

In various embodiments, the method 400 may further include determining an accuracy score based on a distance between the reference point corresponding to the known biological compound the unknown biological sample is identified as and the sample point in the two-dimensional plot formed from a most number of attributes. For example, referring to FIG. 11, the unknown glycan may be identified as the first known glycan 1120 and the reference points corresponding to this first known glycan 1120 include reference points 1114 and 1118. Comparing the first two-dimensional plot 1102 including the reference point 1114 and the further two-dimensional plot 1106 including the reference point 1118, the first two-dimensional plot 1102 is formed from a greater number (Y) of attributes. Therefore, an accuracy score 1124 may be calculated based on a distance 1126 between the reference point 1114 and the sample point 1108 in the first two-dimensional plot 1102. In one example, the accuracy score 1124 may be the distance 1126 as shown in FIG. 11. The method 400 may further include reporting the attributes used to identify the glycan and as shown in FIG. 11, these attributes may be reported as the attributes A₁, A₂, . . . , A_{y} used to form the first two-dimensional plot 1102 containing the greatest number of attributes.

### Example Implementation for the System 300 and the Method 400

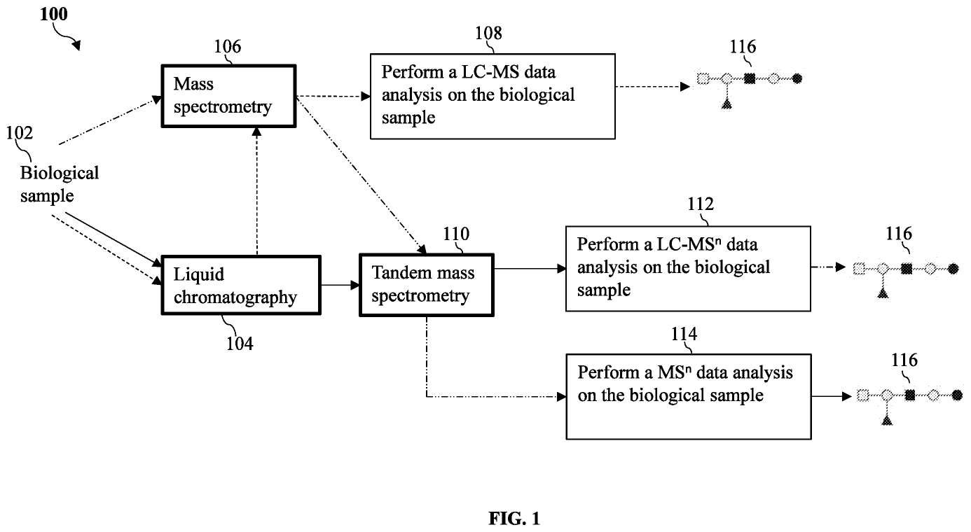

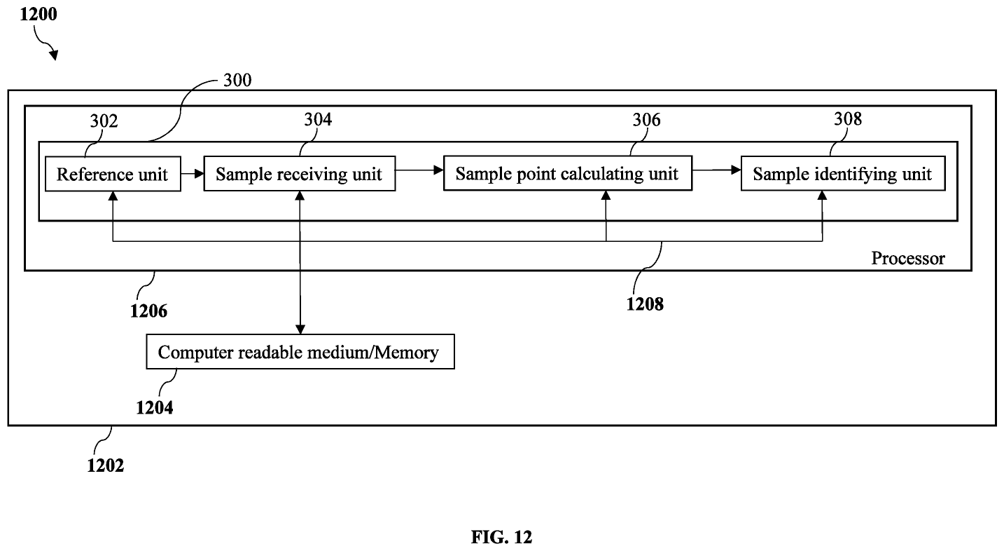

FIG. 12 is a diagram illustrating an example of a hardware implementation for an apparatus 1200 employing a processing system 1202. In one embodiment, the apparatus 1200 may implement the system 300 and method 400 described above in FIGS. 1-11. The processing system 1202 may be implemented with a bus architecture, represented generally by the bus 1208. The bus 1208 may include any number of interconnecting buses and bridges depending on the specific application of the processing system 1202 and the overall design constraints. The bus 1208 may link together various circuits including one or more processors and/or hardware components, represented by the processor 1206 and the computer-readable medium/memory 1204. The bus 1208 may also link various other circuits such as timing sources, peripherals, voltage regulators, and power management circuits, which are well known in the art, and therefore, will not be described any further.

The processing system 1202 may include a processor 1206 coupled to a computer-readable medium/memory 1204. The processor 1206 may be responsible for general processing, including the execution of software stored on the computer-readable medium/memory 1204. The software, when executed by the processor 1206, may cause the processing system 1202 to perform the various functions described supra for any particular apparatus. The computer-readable medium/memory 1204 may also be used for storing data that is manipulated by the processor 1206 when executing software. The processing system 1202 may further include at least one of the reference unit 302, sample receiving unit 304, sample point calculating unit 306 and sample identifying unit 308 of the system 300. These components 302, 304, 306, 308 may be software components running in the processor 1206. Alternatively, they may be resident/stored in the computer readable medium/memory 1204, or may be one or more hardware components coupled to the processor 1206, or some combination thereof.

In various embodiments, a computer program product may be provided. The computer program product may include computer-readable instructions that implement an application for identifying an unknown biological sample. The computer program product may be configured to be executed on one or more computing devices, each having one or more processors. The application may be configured to implement the method 400. For example, the application may be configured to provide a two-dimensional plot comprising a plurality of stored reference points corresponding to respective known biological compounds (similar to that formed in 402 as described above). The application may include instructions for performing 404-408 of method 400.

In various embodiments, a kit may be provided. The kit may include an extraction device for extracting an unknown biological sample, at least one experimental device for determining sample measurements for the extracted unknown biological sample and a computing device configured to execute the above-described computer program product.

In various embodiments, a visualization software may further be provided in the system 300 to provide various functions for the user. The software may be provided as a web application hosted on a server machine, a desktop application or a mobile application device. A clear visualization of the reference measurements of a plurality of known biological compounds (in the form of reference points in a two-dimensional plot) may be achieved via the visualization software. The two-dimensional plot with the reference points may be exported as a high-resolution image. Similarly, the position of a sample point relative to the positions of the reference points on the two-dimensional plot may also be visualized. Using the two-dimensional plot helps to facilitate the identification of the reference point nearest to the sample point (and hence, the known biological compound most similar to the unknown biological sample). The plot with the sample point may also be exported as a high-resolution image. The software may further include interactive features. For example, a user may click on the two-dimensional plot (e.g. click on the reference points) to reveal the known biological compound associated with each reference point. The user may also click on the two-dimensional plot (e.g. click on the reference points) to reveal whether each reference point was generated from reference measurements obtained experimentally or from reference measurements obtained by machine learning. The software may also show comparisons between the sample point and the reference points, e.g. show the distance between the sample point and each reference point and may highlight the reference point nearest to the sample point.

### Example Implementation of Method 400 for Identifying an Unknown Glycan

In one example, 404 to 408 of method 400 was implemented to identify an unknown glycan. Three sample measurements were received for the unknown glycan at 404, and 406 and 408 were implemented with the workflow 1000 as shown in FIG. 10. In this example, multiple two-dimensional plots formed from different numbers of attributes were used and for each plot, 1004, 1012, 1016, 1018 and 1020 of the workflow 1000 were performed in this order.

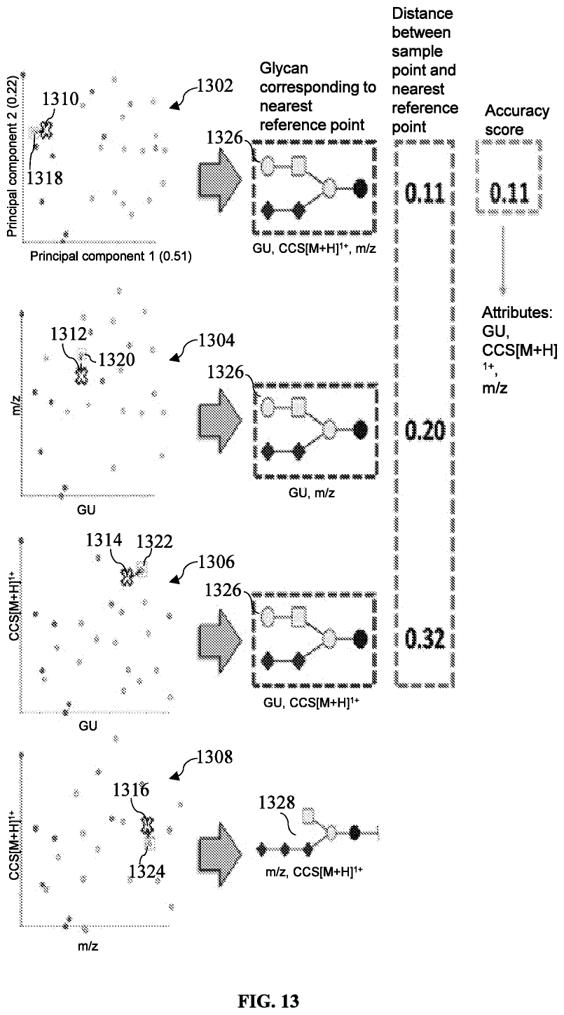

FIG. 13 shows the results obtained from performing 404 to 408 of method 400 on the unknown glycan. As shown in FIG. 13, the multiple two-dimensional plots used in this example included a first two-dimensional plot 1302, and three further two-dimensional plots including a second two-dimensional plot 1304, a third two-dimensional plot 1306 and a fourth two-dimensional plot 1308. The first two-dimensional plot 1302 was formed from three attributes GU, CCS[M+H]¹⁺ and m/z and reference measurements for these three attributes of known glycans were compressed using principal component analysis into the two-dimensional plot 1302. The second two-dimensional plot 1304 was formed from two attributes GU, m/z, the third two-dimensional plot 1306 was formed from two attributes GU, CCS[M+H]¹⁺ and the fourth two-dimensional plot 1308 was formed from two attributes m/z, CCS[M+H]¹⁺. Although not shown in FIG. 13, further two-dimensional plots may be used in this example. For instance, further two-dimensional plots with each formed from a single attribute m/z, GU or CCS[M+H]¹⁺ may be used.

The sample measurements obtained for the unknown glycan were mapped to each of the two-dimensional plots 1302, 1304, 1306. To do so, sample points 1310, 1312, 1314, 1316 were calculated in each of these two-dimensional plots 1302-1308 and the nearest reference point 1318, 1320, 1322, 1324 to each of these sample points 1310, 1312, 1314, 1316 was determined after categorizing the unknown glycan into one of multiple groups of isomers and retaining only the reference points corresponding to this group of isomers. As shown in FIG. 13, the nearest reference points 1318, 1320, 1322 to the sample points 1310, 1312, 1314 in the first, second and third two-dimensional plots 1302, 1304, 1306 correspond to a same glycan 1326, whereas the nearest reference point 1324 to the sample point 1316 in the fourth two-dimensional plot 1308 corresponds to a different glycan 1328. Accordingly, the unknown glycan was identified as the glycan 1326 since the most number of nearest reference points 1318, 1320, 1322 correspond to this glycan 1326.

The distances between the nearest reference points 1318, 1320, 1322 to the sample points 1310, 1312, 1314 in the first, second and third two-dimensional plots 1302, 1304, 1306 were calculated as 0.11, 0.20 and 0.32. Since the first two-dimensional plot 1302 was formed from a greater number of attributes as compared to the second and third two-dimensional plots 1304, 1306, the accuracy score was calculated as the distance between the nearest reference point 1318 to the sample point 1310 in the first two-dimensional plot 1302, in other words, the accuracy score was calculated as 0.11. Further, the attributes used to identify the glycan were reported as GU, [M+H]¹⁺, m/z.

### Example Implementation of Method 400 for Identifying Glycosphingolipid Glycans in Triple Negative Breast Cancer

There are various types of breast cancers including triple positive breast cancers (TPBC) such as the BT474 cell line, estrogen receptor positive breast cancer (such as the MCF-7 cell line) and triple negative breast cancers (TNBCs). TNBCs make up 10-20% of all breast cancers and are difficult to diagnose due to a lack of well-defined markers. Previous glycosylation gene expression analysis has shown three genes mainly involved in O-glycan and GSL glycan metabolism to be diagnostic of the TNBC state as compared to luminal and HER2 breast cancers. Within the TNBC classification itself, there have been up to six different subtypes reported that have previously been successfully stratified by an total gene expression cluster analysis. These include the BT549 cell line and the MDA-MB-453 cell line. The BT549 cell line has been classified as a mesenchymal and basal B subtype, and is considered a non-invasive TNBC while the MDA-MB-453 cell line in comparison is an invasive, luminal androgen receptor and luminal subtype despite displaying an epithelial morphology similar to the BT549 cell line. Limited glycomic profiling has been carried out in human breast cancer models.

The following describes an example process (in sections 1 to 7) for identifying GSL glycans in breast cancer cells, where the process involves an example implementation of method 400.

1. Obtaining and Labelling Known GSL Means (GSL Glycan Standards) and Unknown GSL Means from Breast Cancer Cells

In the example process, known biological compounds in the form of GSL glycan standards (in other words, known glycans) and unknown biological samples in the form of unknown GSL glycan samples from breast cancer cell lines were obtained and labelled in the following manner.

Materials. GSL glycans standards (73 standards covering ganglio-, lacto-, neolacto-, globo- and isoglobo series) were purchased from Elicityl (Crolles, France) and LNFP1 glycan standard from Prozyme (CA, USA). GM2 GSL, Procainamide hydrochloride, sodium cyanoborohydride, polyvinyl pyrrolidone and rEGCase II from Rhodocococcus sp. were purchased from Sigma-Aldrich (MO, USA). PD MiniTrap G-10 SEC cartridges were purchased from GE Life Sciences (IL, USA). Ammonium formate solution was purchased from (Waters, (Milford, USA) and, Procainamide-labelled Dextran Homopolymer from Ludger Ltd. (Oxon, UK). Immobilon-P PVDF membrane (0.45 μm), acetonitrile, DMSO, acetic acid, methanol, 1-butanol, chloroform, sodium acetate and LC-MS grade water were from Merck (NJ, USA). Phosphate Buffered Saline (PBS) was from Axil Scientific (Singapore, Singapore) and polypropylene plates from Corning® Costar® (UT, USA). Rosewell Park Memorial Institute (RPMI) 1640 media, Leibovitz's L-15 media and penicillin-streptomycin were from Gibco (NY, USA) and HyClone™ Fetal Bovine Serum was from FisherScientific (USA). MDA-MB-453, MCF-7 and BT474 cells were purchased from the American Type Culture Collection ATCC (VA, USA) and BT549 cells were from the National Cancer Institute NCl-60 panel (Bethesda, Md.).

Cell Culture and Harvest. In this example, MDA-MB-453, MCF-7, BT474 and BT549 cells were cultured and harvested in the following manner. BT549 cells were cultured in RPMI 1640 media supplemented with 10% Fetal Bovine Serum (FBS) and 1% Penicillin-Streptomycin, and collected at passage number 13. MCF7 cells were cultured in RPMI 1640 media supplemented with 10% Fetal Bovine Serum (FBS), and collected at passage number 16. BT474 cells were grown in 1:1 DMEM:Ham's F12 supplemented with 2 mM L-glutamine and 10% FBS, and collected at passage number 18. The cells were grown to 80% confluency at 37° C. in 5% CO₂. The MDA-MB-453 cell line was cultured in Leibovitz's L-15 media supplemented with 10% FBS, and collected at passage number 7. The cells were grown to 80% confluency, at 37° C. in an atmospheric gas composition. Cells were washed twice with PBS before scraping for collection. Cells were pooled from different culture flasks to make a total of 3×10⁸ cells per triplicate and pelleted by centrifugation at 2500 g for 20 min. Pellets were stored at −80° C.

Extraction of GSLs from Cells. In this example, GSLs were extracted from the breast cancer cells including the MDA-MB-453 cells, MCF-7 cells, BT474 cells and BT549 cells using a modified Folch extraction procedure. This procedure may help to enrich sialylated gangliosides from cell cultures. In particular, five ml of chloroform/methanol (2:1) was added to each cell pellet and left overnight at 4° C. on a spinning tube rotator. The resulting samples were centrifuged at 1800 g for 20 min, and the supernatant was extracted. The pellet was re-extracted and the supernatants were combined, followed by drying under nitrogen gas. Some of the extracted crude GSLs were then purified by n-butanol/water partitioning. The extracted dried GSLs were solubilized in 2 ml of n-butanol/water (1:1), vortexed, and centrifuged at 1000 g for 10 min. The upper butanol and lower aqueous layers were separated into individual glass vials. To the butanol layer, 1 ml of water/n-butanol (10:1) was added and mixed. To the lower aqueous layer, 1 ml of water/n-butanol (1:10) was added and mixed. Both mixtures were then subjected to centrifugation at 1000 g for 10 min. The combined butanol layers were dried under nitrogen gas.

As described above, some of the extracted crude GSLs were purified by n-butanol/water partitioning. This may help to remove polar impurities and reduce the amount of contaminant monosaccharides from crude GSL extracts. Accordingly, including this partitioning process may help to remove contaminating peaks that do not correspond to glycan compositions in the glycan profiles of the GSLs. However, although several of these contaminating peaks may be removed by performing the n-butanol/water partitioning, this partitioning may also greatly affect the peaks corresponding to the GSL glycans.

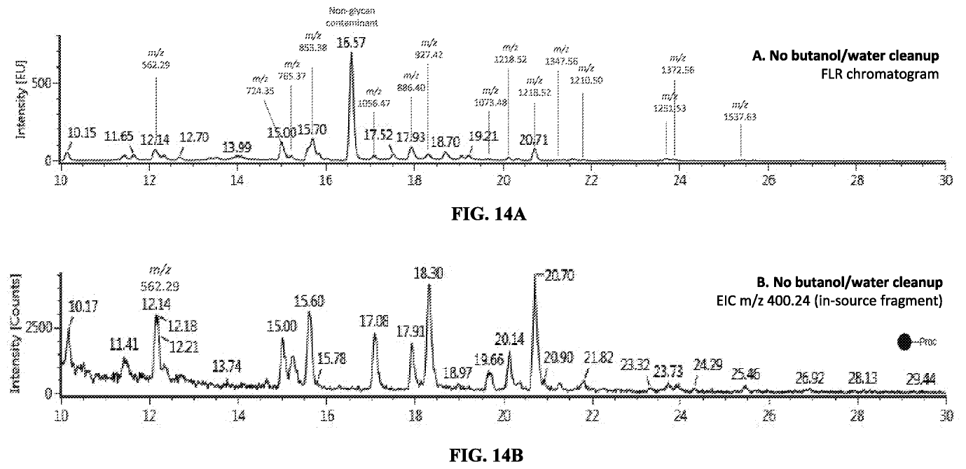

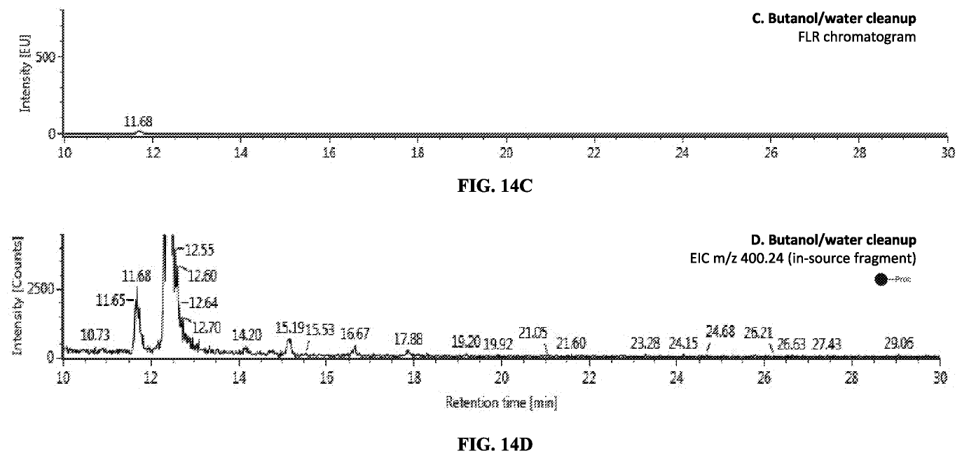

For example, FIGS. 14A and 14B show results obtained with GSL glycans released from GSLs extracted from BT474 breast cancer cells without the n-butanol/water partitioning performed on these extracted GSLs, whereas FIGS. 14C and 14D show results with the n-butanol/water partitioning. In particular, FIGS. 14A and 14C each shows a chromatogram obtained by performing hydrophilic interaction liquid chromatography with fluorescence (HILIC-FLD) on the GSL glycans. In FIG. 14A, the m/z values associated with the compositions of various glycans are also shown. FIGS. 14B and 14D each shows an extracted ion chromatogram (ETC) of a sample with an m/z of 400.24 (in-source fragment of the reducing end Glucose-Proc) from the GSL glycans of FIGS. 14A and 14C respectively. The EICs of FIGS. 14B and 14D can help to differentiate between the peaks corresponding to glycans and those corresponding to non-glycan contaminants in the HILIC-FLD chromatograms of FIGS. 14A and 14C.

As shown in FIGS. 14A and 14B, without the n-butanol/water partitioning, the HILIC-FLD chromatogram includes several peaks, with some peaks corresponding to glycans and some corresponding to non-glycan contaminants. As shown in FIGS. 14C and 14D, with the n-butanol/water partitioning, majority of the peaks (including those corresponding to the glycans) are removed from the HILIC-FLD chromatogram and the EIC. In other words, although the n-butanol/water partitioning can help to remove the peaks corresponding to non-glycan contaminants, it may also cause the loss of peaks corresponding to the glycans. Accordingly, the partitioning process was omitted for several of the GSLs in this example. This can also help to improve the yield and sensitivity of GSLs extracted from the cells.

Glycan Release. In this example, glycans were released from the extracted GSLs using polyvinylidene difluoride (PVDF) membrane-based glycan release and in-solution-based glycan release. In general, PVDF membrane-based glycan release involves the immobilisation of the hydrophobic ceramide portions of GSL glycans to a hydrophobic membrane surface, leaving the hydrophilic glycan portions of the GSL glycans exposed for enzymatic release. In-solution-based glycan release may be used to perform glycan release from glycoconjugates and usually require fewer experimental steps compared to PVDF membrane-based glycan release. In-solution-based glycan release may also produce glycan profiles with double the signal intensity compared to PVDF membrane-based glycan release for glycoprotein N-glycan release.

In this example, for PVDF membrane-based glycan release, 6×10 μg GM2 was solubilized in 50 μL chloroform/methanol (2:1) and spotted onto individual membrane spots in a 96-well polypropylene plate. Samples were left to bind overnight before blocking with 1% PVP in 50% methanol/water. For in-solution-based glycan release, dried GSL samples were solubilized in 50 μL of 50 mM sodium acetate (pH 5.0).

Enzyme amounts and digestion times may affect the types of GSL glycans released, and digestion times ranging from 16 to 48 h may be used for releasing GSL glycans derived from biological samples. In this example, both the PVDF-bound samples and in-solution samples (arising from the PVDF membrane-based glycan release and in-solution-based glycan release respectively) were treated with 4 82 L (8 mU) of rEGCase II and incubated at 37° C. for 18 h for one-night's digestion and two nights' digestion. For the two nights' digestion, samples were incubated initially for 24 h followed by the addition of a further 2 μL (4 mU) of rECGase II, and the samples were incubated for another 19 h. The released glycan solution was transferred to fresh Eppendorf tubes containing 1 mL chloroform/methanol/water (8:4:3). Sample vials were washed with 50 μL of DI water which was pooled in the Eppendorf tube. Tubes were vortexed and centrifuged. The upper glycan-containing methanol/water layer was extracted and dried in a vacuum centrifuge.

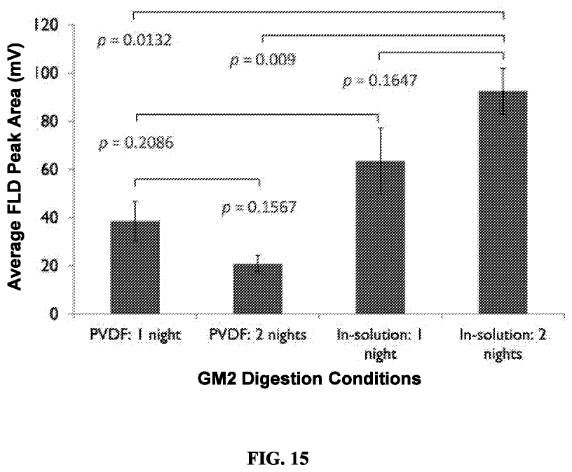

To compare the PVDF membrane-based glycan release against the in-solution-based glycan release, the GM2 standard (GM2 glycan) was released using rEGCase II from Rhodocococcus sp. Fluorescence peak areas for released GM2 glycan using one night's digestion and two nights' digestion with rEGCase II were also compared. FIG. 15 shows results obtained by performing HILIC-UPLC-FLD on the GM2 glycans released using different GM2 digestion conditions. In particular, FIG. 15 shows the average fluorescence (FLD) peak areas obtained for the GM2 digestion conditions including PVDF membrane-based glycan release with one night's digestion (PVDF:1 night), PVDF membrane-based glycan release with two nights' digestion (PVDF:2 nights), in-solution-based glycan release with one night's digestion (In-solution: 1 night) and in-solution-based glycan release with two nights' digestion (In-solution: 2 nights). As shown in FIG. 15, the largest glycan yield was observed with the in-solution-based glycan release with two nights' digestion (in other words, after two night's in-solution digestion). This largest glycan yield was significantly higher (p=0.0132 or 0.009; t-test) than any of the glycan yields obtained with the PVDF membrane-based glycan release.

Fluorescent labelling. In this example, glycans (including both the glycan standards and the glycans released from the breast cancer cells) were labelled with procainamide. In this example, to label the glycans with procainamide, the glycans were solubilised in 10 μL water and transferred to a glass vial for labelling with procainamide via reductive amination. A 100 μL solution of 0.4 M procainamide hydrochloride and 0.9 M sodium cyanoborohydride in 7:3 (v/v) DMSO/acetic acid was prepared, followed by the addition of 30 μL water to result in a clear labelling mixture. Ten microliters (10 μL) of procainamide labelling mix was added to each sample and incubated at 37° C. for 16 h. The glycans may alternatively be labelled with 2-Aminobenzamide (2-AB). To do so, the glycans may be solubilised in 25 μL water and transferred to a glass vial for labelling with the 2-AB via reductive amination. A mixture of 20 μL 0.35 M 2-AB and 1 M sodium cyanoborohydride in 7:3 (v/v) DMSO/acetic acid may be added to each sample and incubated at 37° C. for 16 h with agitation at 800 rpm.

Labelling the glycans with procainamide may help to improve the detection of the glycans in MS techniques (as 2-AB tends to have a poor ionisation efficiency). For example, procainamide can provide effective MS signals suitable for obtaining CCS values of GSL glycans. FIG. 16A shows MS EIC mean peak areas (with total number of peak areas n=5) for a procainamide-labelled LNFP1 pentassacharide sample (LNFP1-Proc) and for a 2-AB labelled LNFP1 pentassacharide sample (LNFP1-2AB). FIG. 16B shows the fluorescence mean peak areas (with total number of peak areas n=5) for a procainamide-labelled LNFP1 pentassacharide sample (LNFP1-Proc) and for a 2-AB labelled LNFP1 pentassacharide sample (LNFP1-2AB). Referring to FIGS. 16A and 16B, it can be seen that using procainamide as the label can achieve a fluorescence level that is 16.6 times higher (p=3.542e-08; t-test) and a MS signal intensity that is 93 times higher (p=1.174e-08; t-test) as compared to using 2-AB as the label. Similar observations have also been made in the analysis of N-glycans.

Post-labelling clean-up. For removal of excess label, the glycan-label mixtures were diluted with water to a total volume of 300 μL and then applied to individual PD MiniTrap G-10 SEC cartridges. For breast cancer glycan samples, 10% of the sample was removed for G-10 clean-up. Glycans were eluted in water and dried in a vacuum centrifuge.

2. Constructing a Multi-Attribute GSL Glycan Library (402 of method 400)

In this example, the 73 GSL glycan standards purchased from Elicityl (Crolles, France) as mentioned above and derived from 36 separate compositions were used to build a multi-attribute GSL glycan library (which is an experimental library in this example).

In particular, in this example, 402a of method 400 (obtaining a plurality of reference measurements experimentally) was implemented by performing a hydrophilic interaction chromatography ultra-high performance liquid chromatography with fluorescence coupled with electrospray ionisation ion mobility mass spectrometry (HILIC-UPLC-FLD ESI-IM-MS) technique on the 73 glycan standards that have been labelled. Details of performing this technique on the 73 glycan standards are provided in section 5 below.

An experimental library was then formed in 402c of method 400 using the experimentally obtained reference measurements from 402a of method 400. The experimental library constructed in this example contains reference measurements for five attributes: theoretical mass, experimentally observed GU and CCS values for three detected ion states or charge states (CCS[M+H]¹⁺, CCS[M+2H]²⁺ and CCS[M+Na]²⁺). Table A1 below shows the experimental library constructed in this example. As shown, Table A1 lists a number of glycans, and their compositions and structures which may be obtained from their product information. Table A1 further lists reference measurements for the following five attributes of each glycan: (1) a theoretical mass in the form of procainamide-labelled neutral mass, (2) an experimentally observed GU value in the form of mean GU±SEM (standard error of the mean) (95% C.I. (confidence interval)), (3) a CCS[M+H]⁺ value (4) a CCS[M+2H]²⁺ value and (5) a CCS[M+Na]²⁺ value. The CCS values in Table A1 are in the form of mean ^{TW}CCS_{N2}(Å²) (nitrogen collisional cross sectional value with units Å²)±SEM (95% C.I.). The procainamide-labelled neutral masses listed in Table A1 are calculated theoretical masses and may fall in a higher range as compared to masses of unlabelled glycans. The GU values and CCS values in Table A1 are reference measurements experimentally obtained from the HILIC-UPLC-FLD ESI-IM-MS technique. For example, the CCS values represent the IM-MS CCS values for the glycan standards. In Table A-1, in the nomenclature ^{TW}CCS_{N2}, the superscripted prefix denotes the measurement type (travelling wave) and the subscripted suffix specifies the drift gas (N2). The structures in the experimental library are representative of different types of glycan structures, namely isoglobo, globo-, neolacto-, lacto-, and ganglioside structures.

402c-402f of method 400 were then implemented and a two-dimensional plot was constructed using the reference measurements of the known glycans in the experimental library in Table A1.

3. Testing Glycan Matching/Assignment with Different Numbers of Attributes Using the Glycan Standards

In this example, the glycan standards were analysed by LC-MS a further six times and sample measurements obtained from these six analyses were treated as sample measurements of unknown glycans (or in other words, “test glycans”/“de-identified glycans”). The sample measurements from the six analyses of the test glycans were searched against the experimental library in various combinations and the degree of accuracy assignment was calculated by bootstrapping the 73 glycan standards, i.e. selecting 80% of the 73 glycan standards at random to search against the library 1000 times.

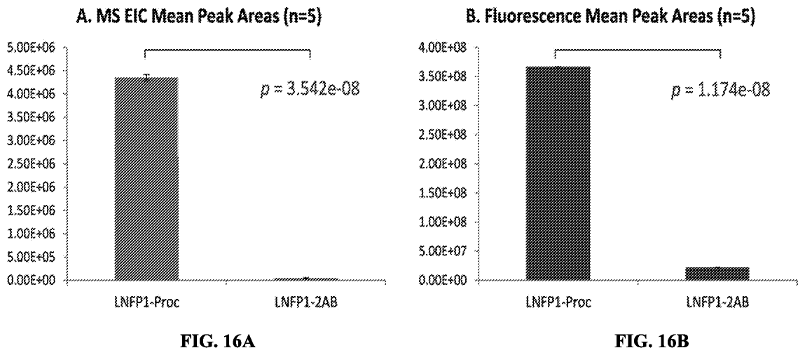

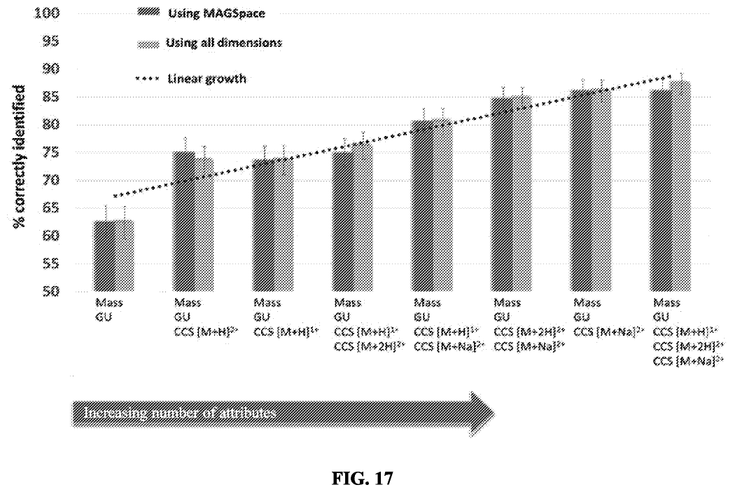

FIG. 17 shows a plot illustrating the percentages of correctly identified glycans when different numbers of attributes are used. In particular, for each number of attributes used, FIG. 17 show the percentages of correctly identified glycans when MAGSpace is used (in other words, when the reference measurements and sample measurements are compressed into reference points and sample points in two-dimensional plots, and are then matched using the reference and sample points in a manner similar to that described above with reference to method 400, with Euclidean distance calculated on the compressed form of the measurements as described in section 6.1 below) and for comparison, when all dimensions are used (in other words, when no compression of the measurements is performed and Euclidean distance is calculated on an uncompressed form of the measurements as elaborated in section 6.2 below).

As shown in FIG. 17, when only the sample measurements for the attributes of observed mass (de-convoluted) and GU were used for searching against the experimental library, only 62.43% of glycan assignments were correct and this was the lowest accuracy observed. However, when sample measurements for mass and GU were used in combination with sample measurements for the attribute CCS, the matching accuracy increased (from 62.43% to 73.60% when using CCS[M+2H]²⁺ values, to 73.65% when using CCS[M+H]¹⁺ values and to 86.09% when using CCS[M+Na]²⁺ values. This indicates that CCS values are helpful glycan identifiers when used in library matching. Of the three ion states, using CCS[M+Na]²⁺ values resulted in the highest glycan matching accuracy, whereas using the CCS[M+H]²⁺ values resulted in the lowest glycan matching accuracy. The latter may be because CCS values for doubly protonated species were the least detected of the three ion states (detected for only 69.8% of the glycan standards in the experimental library in Table A1 as opposed to the CCS[M+H]¹⁺ and CCS[M+Na]²⁺ values which were respectively detected for 94.5% and 98.6% of the glycan standards). By using CCS values for all charge states, particularly doubly protonated states, a glycan matching accuracy greater than 87.42% may be attained. This may be partly achieved through the continuous addition of reference measurements to the library and the incorporation of ESI solvent conditions that increase the efficiency of analyte protonation over sodium adduct formation.

Further, as shown in FIG. 17, using the sample measurements for mass and GU alone resulted in a significantly lower accuracy (p<0.001; t-test) as compared to using sample measurements for three or more attributes. This may be due to the presence of several isometric structures in GSL glycans causing ambiguity in structural assignments. As the number of attributes used for the identification of the test glycans increases, the accuracy of assignment increases linearly. When sample measurements of all five glycan attributes (mass, GU, CCS [M+H]¹⁺, CCS [M+2H]²⁺, CCS [M+Na]²⁺) were used for matching, this resulted in the highest accuracy of assignment (87.42%) and was significantly higher than using the sample measurements for mass and GU alone. Accordingly, FIG. 17 shows that more accurate identification results can be achieved with a greater number of attributes used for the identification. Further, comparing the percentages of correctly identified glycans when MAGSpace was used and when all dimensions were used in FIG. 17, no significant difference (p>0.3; t-test) was observed. In other words, compressing the measurements into a two-dimensional space does not significantly reduce the accuracy of identifying unknown glycans. Accordingly, easier visualization of how the unknown glycans relates to known glycans using the two-dimensional sample and reference points can be achieved without significantly compromising the accuracy of identifying the unknown glycans.

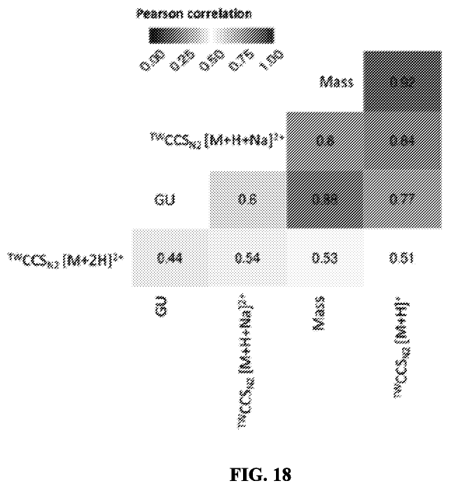

To determine which attribute is responsible for the better separation and discrimination of isomeric glycans, a Pearson correlation analysis was carried out and the results of this analysis are shown in FIG. 18. In particular, FIG. 18 shows correlation coefficients between different attributes, indicating the degree of correlation between the attributes. From FIG. 18, it can be seen that the CCS is an appropriate orthogonal attribute to use as it is not perfectly correlated with either mass or GU and can therefore provide new information. The CCS[M+2H]²⁺ attribute showed the least correlation with all other attributes with correlation coefficients of between 0.44-0.54, indicating that it can provide the greatest isomer discrimination ability.

GSL glycans express a high degree of heterogeneity due to isomerism that may even be higher than that observed in N-glycans of similar masses. The subtle variations in monosaccharide linkages particularly observed in GSL glycan isomers can result in highly similar and overlapping (or in other words, very similar) GU values, thereby increasing the possibility of false positive matches in the library. Isomeric structures can be difficult to distinguish due to their high similarity (same composition but different monosaccharide order or linkage). As the GSL glycan biosynthetic pathway is able to produce a high degree of isomerism (for example, a galactose residue may be linked to the preceding monosaccharide in one of four ways: α-1,3, α-1, 4, β-1,3, β-1,4), the ability to accurately identify isomeric structures can be useful.

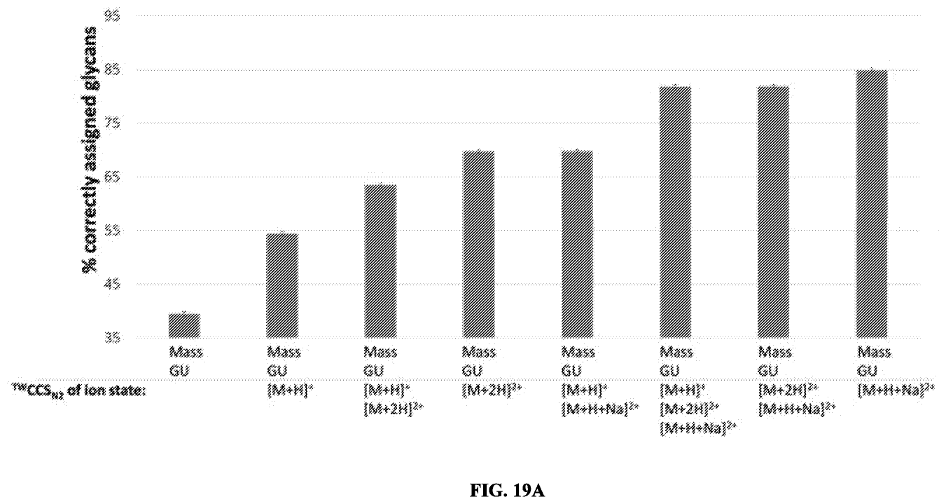

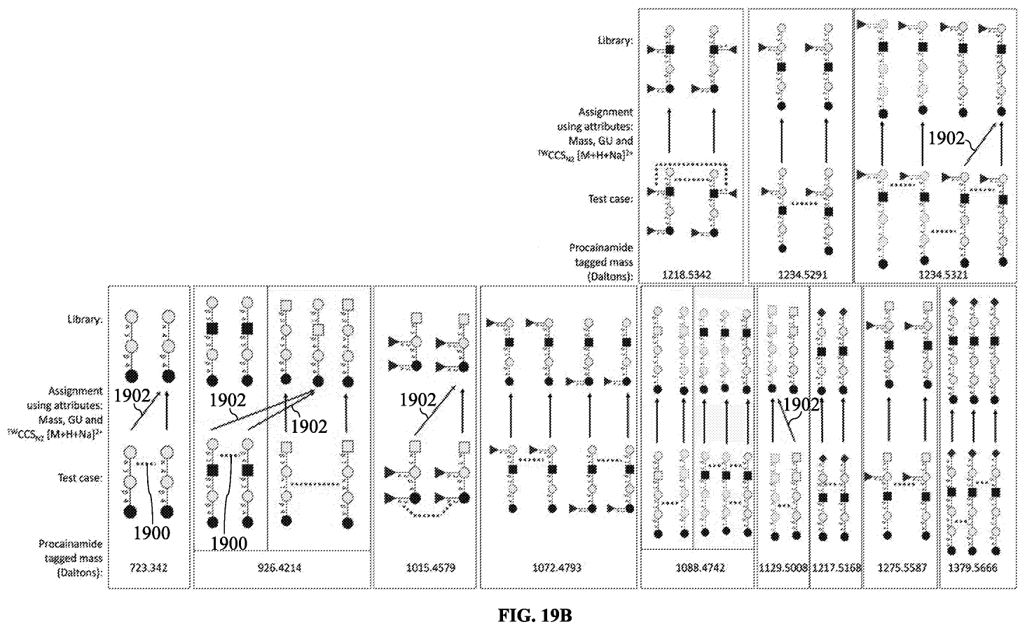

In one example, the experimental library of 73 GSL glycan standards was reduced to 34 glycan standards (containing only isomeric structures) for testing the ability to accurately identify isomeric structures (or in other words, to accurately distinguish glycan monosaccharide linkages) using different numbers of attributes. This reduction was done by removing structures with no isomers or structures that are compositional isomers (isobaric structures) from the experimental library. In this example, each of the remaining 34 glycan standards was used as a test glycan. FIG. 19A shows a bar chart illustrating the average assignment accuracies (in other words, the average accuracies in identifying the 34 glycans) when different combinations of attributes and the aforementioned reduced library (with the 34 glycan standards containing only isomeric structures) were used for the identification. The identification was performed using all dimensions (in other words, using Euclidean distance on an uncompressed form of the measurements as elaborated in section 6.2 below) but similar results may be obtained when using only the MAGSpace since as shown in FIG. 17, the identification accuracies with and without compression of the measurements may be similar. The averages and error bars of the assignment accuracies shown in FIG. 19A were calculated by bootstrapping the 34 glycans in the library. As shown in FIG. 19A, a high assignment accuracy of 84.84% was obtained when using mass, GU, and CCS[M+H+Na]²⁺ attributes. On the other hand, an accuracy of only 39.47% was obtained when using only mass and GU attributes. FIG. 19B shows a visualization of the 34 glycan standards in the library, the 34 test glycans/test cases and the assignments between the 34 test glycans and the 34 glycan standards when the mass, GU and CCS[M+H+Na]²⁺ attributes were used. The procainamide tagged masses listed in FIG. 19B correspond to those listed in Table A1. FIG. 19B further shows the grouping of isomers according to their masses. The dotted lines show areas of monosaccharide linkage differences for each isomer group. As shown in FIG. 19B, 6 out of 34 glycans were wrongly identified/assigned as indicated by arrows 1902, whereas the remaining arrows indicate correct identification of the glycans. Referring to FIG. 19B, visualization of the correct and incorrect assignments made using the mass, GU, and CCS[M+H+Na]²⁺ attributes showed that assignment inaccuracies were not skewed to a particular linkage type. These results also showed that adopting a multi-attribute approach can help improve the accuracy in the identification of the GSL glycans, in comparison to conventional mass and GU matching.

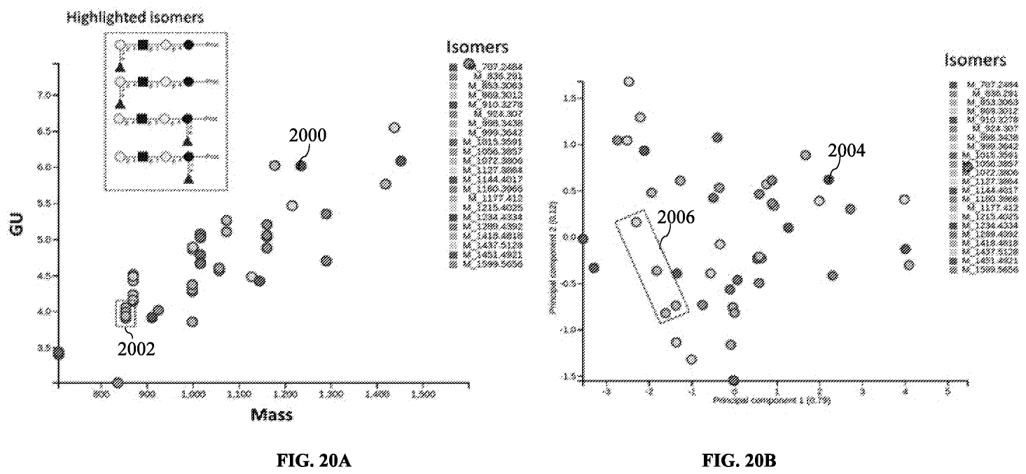

FIG. 20A shows a plot of the GU values against the observed mass measurements for different glycans (each represented by a point e.g. point 2000). The points in the rectangle 2002 correspond to respective glycans in an isomer family of four glycans with a composition of Hexose₃GlcNAc₁Fucose₁-Proc. As shown in FIG. 20A, the GU values of these isomeric glycans are very similar and therefore, there is a high degree of overlap between the points in the rectangle 2002 corresponding to these glycans. FIG. 20B shows a two-dimensional plot including a plurality of points e.g. point 2004 where each point represents a glycan and is formed by compressing measurements of all five attributes (mass, GU, CCS [M+H]¹⁺, CCS [M+2H]²⁺, CCS [M+Na]²⁺) of the glycan into the two-dimensional plot using principal component analysis. In other words, FIG. 20B shows a visualization of all the attributes using principle component analysis. The points in the rectangle 2006 in FIG. 20B correspond to the same isomeric glycans as the points in the rectangle 2002 in FIG. 20A. Comparing these points in FIGS. 20A and 20B, it can be seen that by using all the attributes, there was improved separation between isomeric glycans as compared to using only the mass and GU attributes. In particular, there was complete separation between the points representing the isomeric glycans when plotted in the multi-attribute two-dimensional plot in FIG. 20B.

As described above, FIG. 20B shows the ability of multiple attributes to produce glycan identifiers that are single unique points in a 2-dimensional space. Further, as mentioned above, comparing the percentages of correctly identified glycans when MAGSpace is used and when all dimensions are used in FIG. 17, no significant difference (p>0.3; t-test) was observed. In other words, compressing the measurements into a two-dimensional space does not significantly reduce the accuracy of identifying unknown glycans.

The assignment accuracies described thus far involved the use of a defined library and de-identified glycan standards. The probability of correctly identifying an unknown glycan given a distance for the unknown glycan (in other words, given a Euclidean distance between the sample measurements of the unknown glycan and the reference measurements of the known glycan the unknown glycan is identified as) may be calculated using these assignment accuracies. In one example, the assignment accuracies (percentages of correctly identified glycans) obtained using all dimensions as shown in FIG. 17 were used for a nonlinear regression analysis of accuracy versus distance when using each of the combinations of attributes shown in FIG. 17.

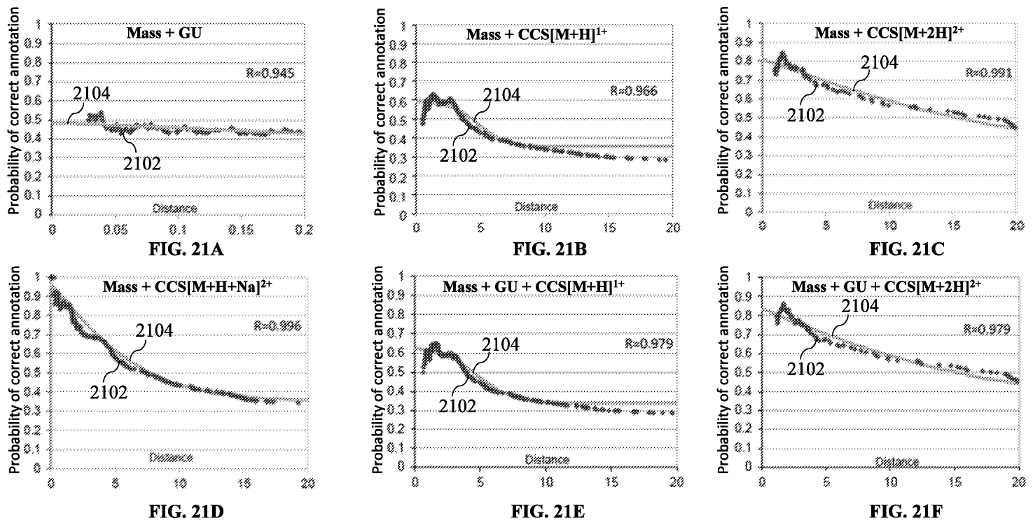

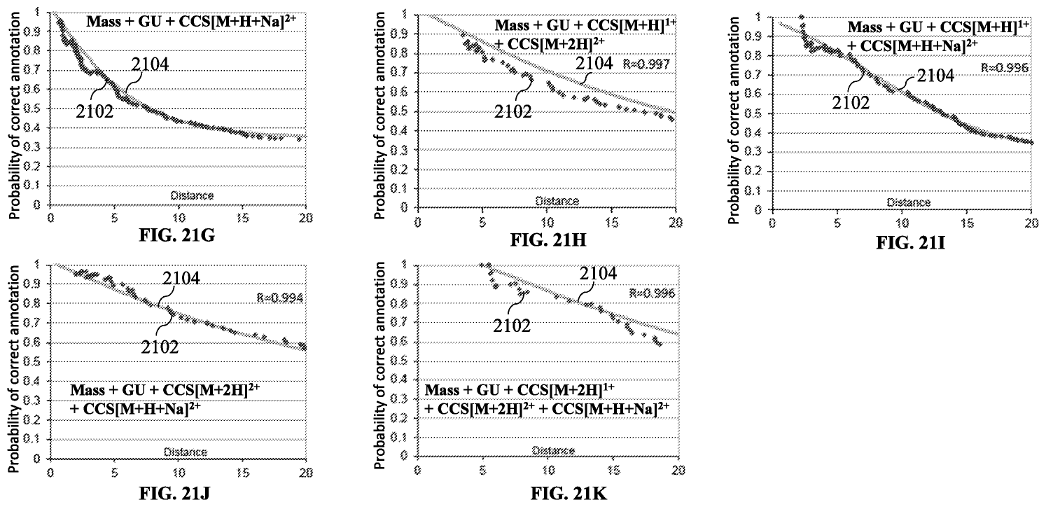

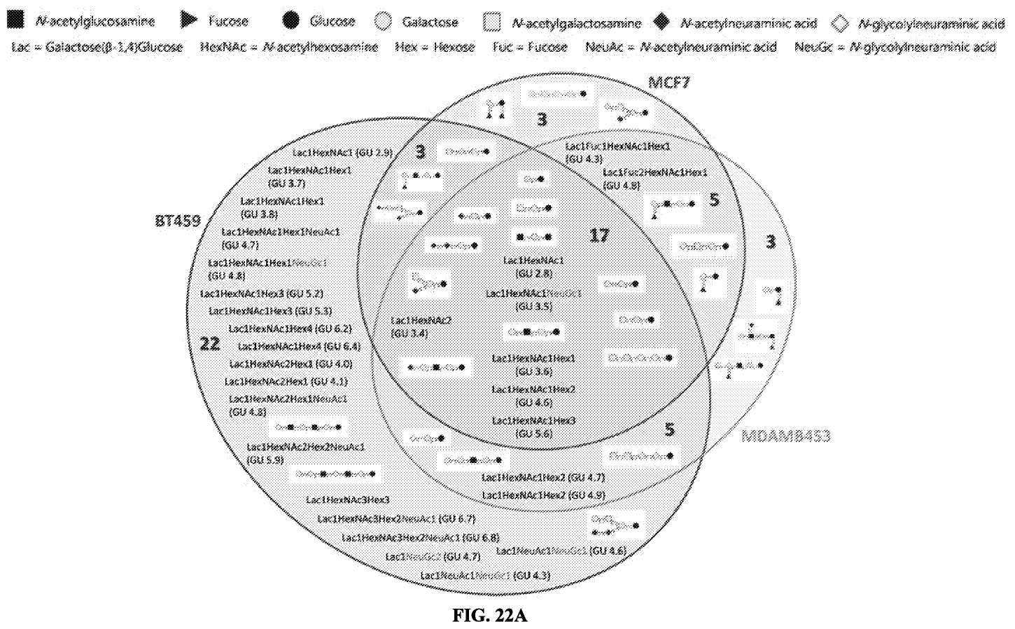

FIGS. 21A to 21K show plots corresponding to respective combinations of attributes shown in FIG. 17. In particular, each of FIGS. 21A to 21K shows a plurality of points 2102, where each point 2102 indicates the proportion of unknown glycans identified correctly out of all the unknown glycans identified with a distance of a particular value. This proportion also represents the probability that an unknown glycan is identified correctly (probability of correct annotation) when the distance for the unknown glycan is of the particular value. Each of FIGS. 21A to 21K further shows a regression curve 2104 formed using the points 2102. In FIGS. 21A to 21K, the ratio “R” represents the coefficient of determination for the plot and as shown by the values of “R”, there is a high correlation between the calculated distances and the accuracies in identifying the unknown glycans. The resulting regression curves 2104 may thus allow the distance for an unknown glycan to be used to calculate the probability that its assignment/identification using multi-attribute matching is correct. A value for the distance associated with a high confidence that the unknown glycan is identified correctly may also be set using the regression curves 2104. The distances used for forming the plots in FIGS. 21A to 21K may be Euclidean distances calculated on uncompressed form of measurements (e.g. in the manner as described in section 6.2 below). However, in various embodiments, instead of using these Euclidean distances, the Euclidean distances calculated in method 400 on compressed forms of the measurements (e.g. in the manner as described in section 6.1 below) may be used to form the regression curves in a similar manner. These regression curves formed with Euclidean distances calculated on compressed forms of the measurements may be similar to those shown in FIGS. 21A to 21K since the identification accuracies of the unknown glycans may be similar with and without compression of the measurements. In one example, the regression curves, such as the curves 2104 or those formed using the distances calculated from method 400, may be used to calculate the probability that the GSL glycans identified in the breast cancer cell lines were correctly identified (as will be elaborated below).

4. Example Implementation of 404-406 of Method 400 to Identify Unknown Glycans from Breast Cancer Cells

As described above in section 1, glycans were extracted from breast cancer cells. As GSL glycosylation changes have been described in ovarian and colon cancers, in this example, GSL glycan differences were characterised in two different TNBC subtypes (BT549 cell line and MDA-MB-453 cell line) with a TPBC subtype (MCF7 cell line) as a non-TNBC control. In this example, 404 of method 400 was implemented by performing the HILIC-UHPLC-FLD ESI-IM-MS technique on the glycans extracted from the breast cancer cells. Sample measurements for the attributes listed in Table A1 were thus obtained. Details of this implementation are provided below in section 5.

Some of the extracted glycans were identified by composition only, whereas others were identified with 404-408 of method 400. In particular, for the glycans identified with method 400, sample measurements for these glycans were obtained at 404 and were used to calculate, at 406, a sample point in the two-dimensional plot constructed with the reference measurements in the experimental library shown in Table A1 (as discussed in section 2). The unknown glycans were then identified at 408 of method 400 using the above-described processes.

In this example, a total of 58 different GSL glycan head-groups (in other words, 58 different GSL glycans/glycan structures) were identified. 47 of the 58 structures were identified in BT549 cells, 30 of the 58 structures were identified in MDA-MB-453 cells, and 28 of the 58 structures were identified in MCF7 cells. 25 of the 58 structures were identified by matching against the glycan experimental library (in Table A1 below) using 404-408 of method 400, and the accuracy scores (or in other words, the average glycan identification distances) were between 0.0165 and 0.4460. The remaining 33 structures were identified by composition only. The structural types detected included ganglio-, globo-, lacto- and neolacto-series (as shown in Table A2).

For each glycan identified from the breast cancer cells using the experimental library with 404-408 of method 400, the probability of correct assignment (given a distance), in other words, the probability that the glycan was correctly identified given a distance for the glycan, was calculated using regression curves formed with Euclidean distances calculated on compressed forms of the measurements, which may be similar to the regression curves 2104 in FIGS. 21A to 21K. For each glycan, a probability was calculated for the case where only mass and GU were used for identifying the glycan (mass and GU based matching) and further probabilities were calculated for the cases where multiple attributes were used for identifying the glycan (multi-attribute matching). The regression curve 2104 used to calculate each probability was chosen based on the attributes used for identifying the glycan. The probabilities obtained for multi-attribute matching (0.6-1) were found to be higher as compared to the probabilities obtained for mass and GU based matching (<0.5) for all glycans. This showed that a higher confidence in glycan assignment may be achieved when using multiple attributes.

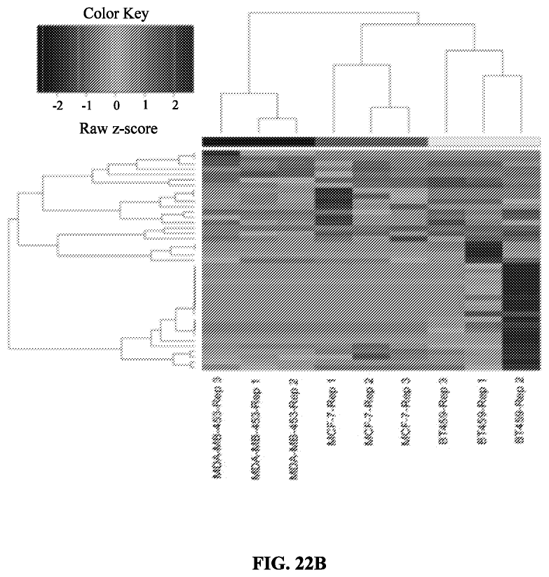

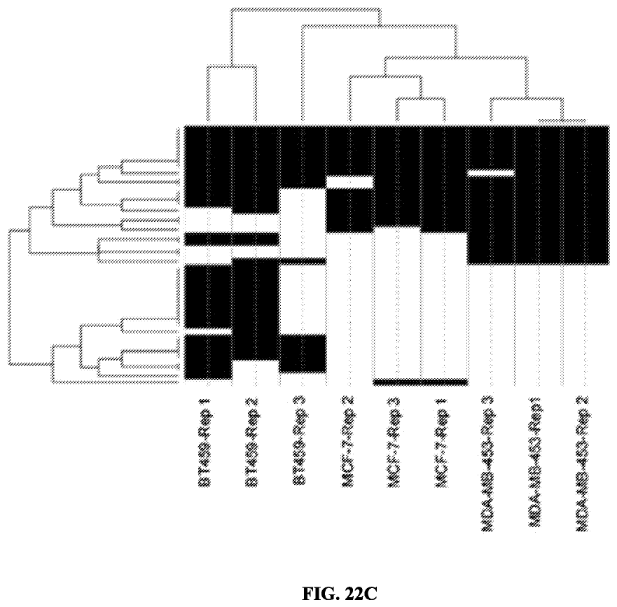

In this example, the 58 identified glycans were derived from 48 liquid chromatography fluorescent (LC-FLD) peaks due to co-elution. Comparison of these peaks using clustering analysis of the relative percentage peak areas (based on FLD as shown in Table A3 below) showed GSL glycan signatures for each cell line. However, as peak components were not uniform across cell types, (e.g., peak 23 contained two glycans in BT459 cells, three glycans in MDA-MB-453 cells, and two glycans in MCF7 cells as shown in Table A2), the peaks were not directly comparable. According, a qualitative comparison was instead performed to compare all the identified glycans. FIG. 22A shows a proportional Venn diagram illustrating a qualitative comparison of the GSL glycans detected in BT549, MDA-MB -453 and MCF7 cells. As shown in FIG. 22A, majority of fucosylated structures (seven out of nine) were detected in the MDA-MB-453 cells. N-Acetylneuraminic (NeuAc) and N-glycolylneuraminic (NeuGc) acid sialylation were observed in all cell types; however, BT459 cells displayed the highest number of sialylated structures and was the only cell type with structures carrying both NeuAc and NeuGc (LacNeuAc1-NeuGc1 isomers at Gus 4.3 and 4.6).

Previously reported glycomic analysis has shown the N-glycomes of MDA-MB-453 and BT549 cytosolic glycoproteins to cluster together away from a normal epithelial cell line. However, the analysis did not show much distinction between the two cancerous cell lines. Minimal stratification in the N-glycomes of membrane glycoproteins of these two cell lines was observed, whilst some differences were observed in the O-glycomes of the membrane glycoproteins of these two cell lines.

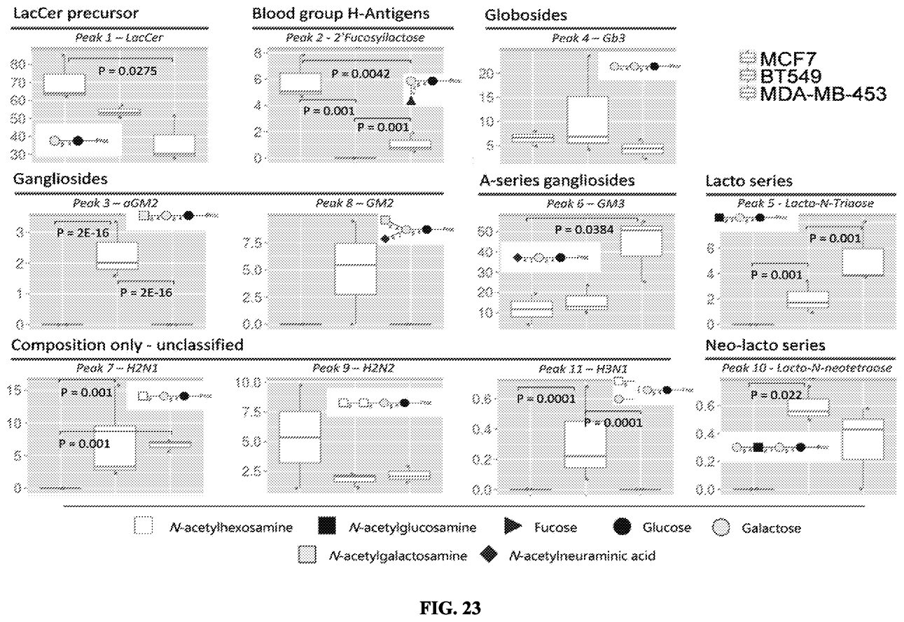

FIG. 22B shows a clustering analysis of LC-FLD peak average relative abundances of 33 peaks commonly detected in MDA-MB-453, MCF7 and BT459 cells analysed in triplicate. As shown in FIG. 22B, distinct glycosylation signatures are present for each cell line. The peak numbers of the clustering analysis in FIG. 22B correspond to those listed in Table A3 below and the z-score denotes normalisation of the relative abundances to a mean that is equal to zero and a standard deviation that is equal to one. FIG. 22C shows a clustering analysis of glycomes based on the presence/absence of glycans in a cell as determined using 404-408 of method 400. As shown in FIG. 22C, distinct GSL glycosylation signatures particularly for MCF7 and MDA-MB-453 cells are present. In the clustering analyses shown in both FIG. 22B and FIG. 22C, the MDA-MB-453 cell line clustered with the MCF7 cell line (despite having countering triple negative statuses) and away from BT459 cell line. In other words, clustering of the glycan relative percentage abundances showed a distinct GSL glycan signature for each cell line in addition to clustering of the two TNBC cell lines away from the MCF7 control. It was also found that the amount of GSL glycan precursor, LacCer was the highest in MCF7 cells, indicating the least glycan processing and extension in this non-TNBC cell line. Between the two TNBC cell lines, significant differences in individual glycan head-groups (Lacto-N-triaose, aGM2 and 2′Fucosyllactose) were also observed. This can be seen in FIG. 23 which shows the average relative glycan abundance of glycans detected in the MCF7, BT549 and MDA-MB-453 cell lines. Accordingly, GSL glycan profiling using method 400 may more effectively to differentiate the different breast cancer cell lines and classify TNBC subtypes. Using method 400, the resulting GSL glycan signatures may allow stratification of TNBC subtypes and may provide an important future diagnostic tool in clinical settings.

### 5. HILIC-UPLC-FLD ESI-IM-MS

HILIC-UPLC-FLD In this example, the labelled GSL glycans (glycan standards and unknown glycans obtained from the breast cancer cells) were analysed by HILIC-UPLC-FLD on an ACQUITY UPLC H-Class (Waters Corporation, MA, USA) with a fluorescence detector. In this example, the chromatography analyses were carried out in the following manner. Dried glycans and dextran were re-solubilised in 88% acetonitrile/12% water and separated at a temperature of 40° C. using an ACQUITY UPLC® BEH-Glycan column (1.7 μm, 2.1×150 mm). Gradient conditions were as follows: 12 to 47% (v/v) 50 mM ammonium formate pH 4.4 in acetonitrile at a flow rate of 0.56 ml/min from 0-36 min, followed by 47 to 70% (v/v) at 0.25 ml/min from 39.5 to 42.0 min. In this example, LNFP1 and GM2 glycans were also analysed at 30° C. with a flow rate of 0.4 ml/min and gradient conditions of 30 to 47% (v/v) 50 mM ammonium formate pH 4.4 in acetonitrile from 0-34.8 min, followed by 47 to 80% (v/v) from 34.8 to 36.0 min. The injection amounts were: 500 fmol for each GSL glycan standard, 7% of breast cancer cell samples, and for GM2 glycan, the equivalent of 25 pmol of GM2 GSL was injected. Fluorescence detection was used for glycan quantitation (λex=310 nm, λem=370 nm for procainamide; λex=330 nm, λem=428 nm for 2-AB).

ESI-IM-MS IM-MS measurements were made online using a Synapt G2S quadrupole/IMS/orthogonal acceleration time-of-flight MS instrument (Waters, Mass., USA) fitted with an electrospray ionization (ESI) ion source. In this example, samples were analysed in resolution mode and mobility separation performed in a traveling-wave drift tube. Spectra were acquired in positive ion mode with a full MS scan over a range of m/z 350-2000 and accumulation time of 1 s. The instrument conditions were as follows: 2.4 kV electrospray ionisation capillary voltage, 15 V cone voltage, 100° C. ion source temperature, 350° C. desolvation temperature, 850 L/hr desolvation gas flow, 40 L/hr cone gas flow, 650 m/s IMS T-wave velocity, and 40 V T-wave peak height. The T-wave mobility gas was nitrogen (N₂) and was operated at a pressure of 3 mbar. The mobility cell was calibrated with Waters Major Mix IMS/Tof Calibration mix. Data acquisition was carried out using MassLynx™ (version 4.1).

To construct the experimental library in this example, the 73 glycan standards were analysed by the HILIC-UPLC-FLD ESI-IM-MS technique on eight separate occasions and the data from these analyses were used as the reference measurements and stored in the experimental library. Analyses were conducted in triplicate and repeated on separate days to calculate a representative average and standard error value of each measurement. CCS values can be influenced by ionisation polarity and adduction, making it possible to observe multiple CCS values for the same glycan present in various ion states. GU values were collected for all 73 structures, whereas for the various charge states: CCS[M+H]¹⁺ values were collected for 68 structures (93.2% of the 73 structures), CCS[M+2H]²⁺ values were collected for 51 structures (69.8% of the 73 structures), and CCS[M+Na]²⁺ were collected for 71 structures (97.3% of the 73 structures). In this example, the formation of sodium adducts was used during positive ion mode ESI to collect ^{TW}CCS_{N2} values for an additional ion state without creating adducts through doping of samples with sodium or lithium salts.

Data Processing. The MassLynx data was imported into the Waters UNIFI Scientific Information System for GU calculation using the ‘Glycan Assay (FLD with MS Confirmation)’ processing method. GU values were calculated by normalising glycan retention times against procainamide-labelled dextran ladder using a fifth order polynomial distribution curve. Mobility data was processed for CCS values calculation using UNIFI's Accurate Mass Screening on IMS data method. Fluorescence (FLD) peak integration was done manually for the area-under-curve based quantitation, and all glycan peak areas within a sample were normalized to 1001 for relative quantitation.

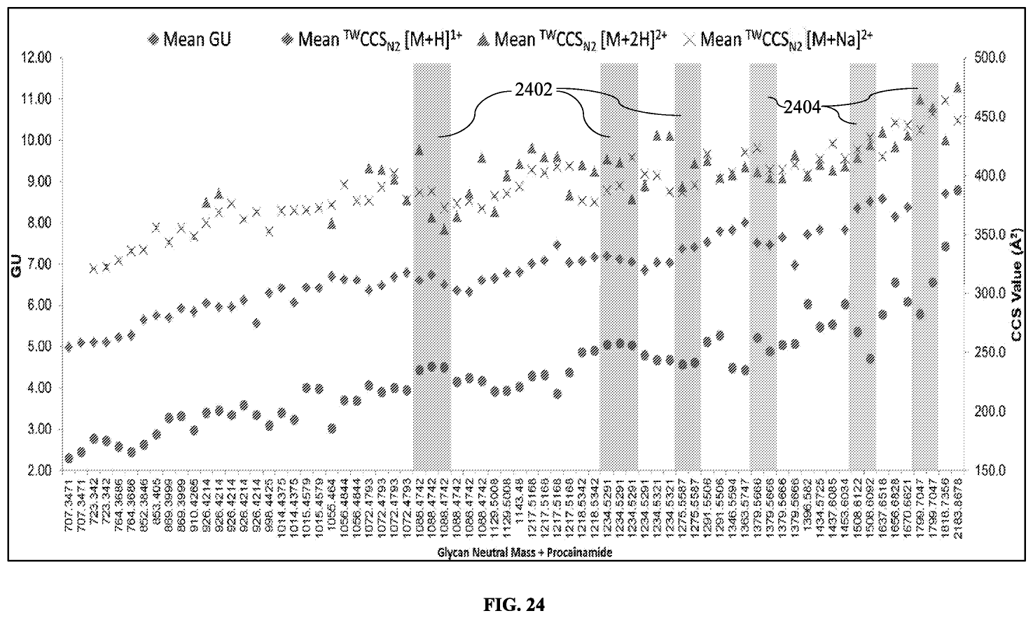

FIG. 24 shows a plot illustrating the reference measurements in the experimental library in Table A1. As illustrated by the panels 2402 of FIG. 24, for some structural isomers, GU values were found to be highly similar making it difficult to distinguish isomers using this attribute alone. On the other hand, greater differences in the ^{TW}CCS_{N2} values of these structural isomers were obtained within the panels 2402. In other words, using the CCS attribute can allow the isomers to be better distinguished. However, referring to the panels 2404 of FIG. 24, in the upper mass range of the library, the reverse was observed where GU values provided greater discriminating power for isomers compared to the ^{TW}CCS_{N2} values. In other words, FIG. 24 shows that using the GU and CCS attributes together can further improve the identification accuracy for all the 73 GSLs.

In the above-described example, sample measurements including GU values, m/z and CCS values were extracted for each glycan peak corresponding to an unknown glycan extracted from the breast cancer cells and these sample measurements were searched against the multi-attribute experimental library using 406-408 of method 400. For cases where the sample measurements were not sufficiently close to the reference measurements found in the library (or in other words, no matching glycan was found in the library), the unknown glycan was identified by composition only (instead of by permuting the detected m/z values to derive all possible GSL glycan structures). All assignments were confirmed manually.

### 6. Using Euclidean Distance as a Similarity Measure

In various embodiments, the sample measurements including GU values, m/z and CCS values of unknown glycans may be searched against the reference measurements of known glycans in the multi-attribute experimental library using Euclidean distance as a similarity measure. In various embodiments, the Euclidean distance may be calculated on a compressed form of the measurements of the attributes. For example, in method 400, the identification of the unknown biological compound and the accuracy score may be determined using Euclidean distances between the sample and reference points, with these points formed from compression of the sample and reference measurements into a two-dimensional space. For comparison, in the above-described examples, Euclidean distances were also calculated on an uncompressed form of the measurements of the attributes. As mentioned above, as shown in FIG. 17, the accuracies in identifying the unknown glycans with and without compressing the measurements were not statistically different (p>0.3). Accordingly, the results obtained in the identification of unknown glycans from the breast cancer cells in the examples mentioned above were similar regardless of whether compression of the measurements was performed.

6.1 Calculating Euclidean Distance on a Compressed form of Measurements

In various embodiments, Euclidean distance may be calculated on a compressed form of measurements in the following manner.

Given a library with N library glycans where each library glycan is associated with k reference measurements G(i)={g₁ⁱ, . . . , gₖⁱ}, the k reference measurements G(i)={g₁ⁱ, . . . , gₖⁱ} for the i^{th} library glycan can be compressed to a two dimensional point (i^{th} reference point) CG(i)={cg₁ⁱ, cg₂ⁱ} using a compression algorithm such as principal component analysis as shown in FIG. 8. Using the same compression algorithm, n sample measurements U=, {u₁, . . . , uₙ} of an unknown glycan can also be compressed to a two dimensional point (sample point) C={c₁, c₂}. The Euclidean distance d²(CG (i), C) between the sample point and the i^{th} reference point (in other words, between the unknown glycan and the i^{th} library glycan) can be computed in the 2 dimensions as d²(CG(i),C)=\sqrt{(c₁−cg₁ⁱ)²+(c₂−cg₂ⁱ)²} where c₁, c₂, are the compressed n sample measurements and cg₁ⁱ, cg₂ⁱ are the compressed k reference measurements for the i^{th} library glycan.

As described above, to identify the unknown glycan, the minimum distance between the compressed sample measurements (sample point) C={c₁, c₂} of the unknown glycan and the compressed reference measurements (reference points) of the library glycans in a same group of isomers as the unknown glycan (e.g. as determined based on the m/z value of the unknown glycan) may be calculated as dₘᵢₙ(C)=min{d²(CG (1), C), . . . , d²(CG (N), C)} where N is the number of library glycans in the same group of isomers as the unknown glycan and dₘᵢₙ (C) is a real number.

6.2 Calculating Euclidean Distance on an Uncompressed form of Measurements

As mentioned above, for comparison of the accuracies in identifying unknown glycans with and without compression of the measurements, Euclidean distance were also calculated on an uncompressed form of measurements in the above-described examples. This was performed in the following manner.

Given an unknown glycan with n sample measurements U={u₁, . . . , uₙ} for n attributes, the distance dⁿ(G(i), U) between the i^{th} library glycan (with k reference measurements G(i)={g₁ⁱ, . . . , gₖⁱ}) and the unknown glycan was computed if n=k. In particular, this distance was computed as dⁿ(G(i), U)=\sqrt{Σₐ₌₁ⁿ(uₐ−gₐⁱ)²} where uₐ, and gₐⁱ are the measurements for the same attribute.

To identify the unknown glycan, the minimum distance between the sample measurements U={u₁, . . . . , uₙ} of the unknown glycan and the reference measurements of the library glycans in a same group of isomers as the unknown glycan (e.g. as determined based on the m/z value of the unknown glycan) was calculated as dₘᵢₙ(U)=min{dⁿ(G (1), U), . . . , dⁿ(G (N), U)} where N is the number of library glycans in the same group of isomers as the unknown glycan and dₘᵢₙ(U) is a real number.

6.3 Forming Reduced Libraries when Sample Measurements of Some Attributes are Unavailable

In some cases, sample measurements of some attributes may be unavailable. In these cases, reduced libraries with reference measurements from different combinations of attributes may be formed from the experimental library and may then be used to identify the unknown glycan. For example, eight libraries may be formed using reference measurements of the following eight combinations of attributes: (1)m/z, GU, (2)m/z, GU, CCS[M+H]¹⁺, (3)m/z, GU, CCS[M+2H]²⁺, (4)m/z, GU, CCS[M+H+Na]²⁺, (5)m/z, GU, CCS[M+H]¹⁺, CCS[M+H+Na]²+, (6)m/z, GU, CCS[M+2H]²⁺, CCS[M+H+Na]²⁺, (7) m/z, GU, CCS[M+H]¹⁺, CCS[M+2H]²+, (8)m/z, GU, CCS[M+H]¹⁺, CCS[M+2H]²⁺, CCS[M+H+Na]²⁺. A minimum distance dₘᵢₙ(C) or dₘᵢₙ(U) may then be calculated using each library having reference measurements of attributes for which sample measurements are available. For instance, when sample measurements for four attributes are available, a minimum distance may be calculated for each of four libraries. In one example, sample measurements for m/z, GU, CCS[M+2H]²⁺, CCS[M+H+Na]²⁺ are available and a minimum distance may be calculated for each of the above-mentioned libraries (1), (3), (4) and (6). When sample measurements for three attributes are available, a minimum distance may be calculated for each of two libraries. In one example, sample measurements for m/z, GU, CCS[M+2H]²⁺ are available and the minimum distance may be calculated for each of the above-mentioned libraries (1) and (3). The minimum distance for each library may be calculated in a manner similar to that described above. For each reduced library, the library glycan corresponding to the calculated minimum distance may be identified, and the unknown glycan may then be identified as the library glycan identified in majority of the reduced libraries.

### 7. Statistics, Clustering and Visualization

In the above-described example, to visualise the glycan attributes of GU, Mass, ^{TW}CCS_{N2}[M+H]¹⁺, ^{TW}CCS_{N2}[M+2H]²⁺ and ^{TW}CCS_{N2} [M+Na]²⁺ in two-dimensional plots, a principle component analysis was carried out. Further, pearson correlation coefficients were calculated. For breast cancer cell line profiling, all glycan assignments were confirmed manually and the probabilities that the glycans were correctly assigned/identified were determined based on the calculated minimum distances and the regression analyses performed using the test glycans described in section 3. Further, only glycans detected in two out of three replicates were kept for further analysis. For hierarchical clustering of breast cancer glycans, peak areas were normalized using z-score which standardizes the peak relative abundances to a mean 0 and a standard deviation 1 and a hierarchy of clusters was built using the complete-linkage algorithm. All p-values reported were found using a Student's paired t-test (assuming normal distribution).

Prior art approaches tend to use either one or at most two attributes to computationally identify glycans. These approaches usually use samples containing few isomeric or isobaric glycans and are able to achieve results that indicate that using only one or two attributes is sufficient for identifying unknown glycans. In view of such results, the limited number of attempts to use more than two attributes and the potentially significant increase in computational complexity when more attributes are used, there has been little motivation to increase the number of attributes used to identify unknown glycans.

However, as described above, in various embodiments, the system 300 may be a useful visualization and precise characterization tool for identifying unknown biological samples such as glycans. This tool may use multi-attribute descriptors from a combination of analytic instrumentation and may allow an automated processing of multi-attribute data to identify unknown samples and may also allow the visualization of large libraries. By “automated”, it is meant that although human interaction may initiate the method (e.g. method 400), human interaction may not be required while the method is carried out (although method 400 may, in some embodiments, be performed semi-automatically, in which case there may be human interaction with the system (e.g. system 300) during the processing).

As described above, the system 300 in the embodiments may use measurements from more than two attributes that are obtained using complex combinations of instrumentation (e.g. LC-IM-MSⁿ). Using measurements from more than two attributes to identify unknown biological samples (such as unknown glycans/glycan conjugates) can help increase the accuracy and speed of identifying these glycans. Using more than two attributes can also improve the accuracy in the identification of isomeric or co-eluting structures as compared to prior art approaches using only one or two attributes.

In various embodiments, the measurements for multiple attributes may be compressed into points in two-dimensional spaces/plots termed MAGSpaces. These points may then be used to identify the unknown samples. By using a two-dimensional plot as compared to a representation with a greater number of dimensions, entire libraries of known biological compounds can be more clearly and easily visualized on for example, a computer screen. Further, the inventors of this application have found that the accuracy in identifying an unknown biological sample using a two-dimensional plot having stored reference points calculated from measurements of more than two attributes is similar to the accuracy obtained using more than two dimensions. This is for example shown in FIG. 17 where the accuracies obtained when using the two-dimensional plot are similar to the accuracies obtained when using more than two dimensions. Accordingly, the computational complexity when using the system 300 may remain low even with the use of multiple attributes to achieve a greater accuracy in identifying the unknown biological sample. Therefore, data generated from big data experiments where measurements for many attributes may be obtained in a single analysis can be effectively used by the system 300.

Further, the embodiments as described above may include an in silico predictive feature. In the embodiments, the library may be expanded to include an in silico library with predicted measurements. This can increase the chances of finding an accurate match for an unknown biological sample.

Further, separation and analytical technologies are advancing at a fast rate and new tools are being developed to obtain measurements for attributes which were previously difficult to obtain. With these measurements and the associated newly characterised known compounds, the libraries used in the system 300 may be updated and the MAGSpaces may be dynamically redefined. In other words, the system 300 may have the ability to easily incorporate output from future technologies and thus, the accuracy in identifying unknown biological samples with this system 300 may be constantly improved with the emergence of the new tools.

In various embodiments, the method 400 may be used in the glycoanalytics field. Embodiments of the present invention may allow reliable screening or diagnosis of GSL-related diseases (such as TNBC as described above) and identification of potential antibody targets. As described above, the method 400 has been demonstrated using data from a database of glycans (Table A1) in the form of an experimental library including reference measurements for glycan standards. However, the method 400 may also be used in other fields in biochemistry or may be extended to the data sciences industry where measurements for multiple attributes may be obtained. For example, the method 400 may be used in the bioprocessing industry to achieve fast, enzyme free, glycan identification (or in other words, annotation) and/or relative abundance measurements of monoclonal antibodies. In various embodiments, the method 400 has also been demonstrated using data from a database of glycans shown in Table A4 below where the glycans in Table A4 correspond to known N-glycans and the reference measurements in Table A4 are obtained from RapiFluor-MS (RFMS)-labelled N-glycans from a monoclonal antibody.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A method for identifying an unknown biological sample, the method comprising:
receiving more than two sample measurements for the unknown biological sample, each sample measurement for an attribute of the unknown biological sample;
calculating a sample point in a two-dimensional plot from the more than two sample measurements for the unknown biological sample;
wherein the two-dimensional plot comprises a plurality of stored reference points corresponding to respective known biological compounds and wherein each reference point is calculated from a plurality of reference measurements for more than two attributes of the corresponding known biological compound, each attribute being different from another attribute; and
identifying the unknown biological sample by comparing the sample point against the plurality of reference points in the two-dimensional plot.

2. The method according to claim 1,
(i) wherein each reference measurement is obtained experimentally or by a machine learning algorithm, or
(ii) wherein the reference measurements for at least one known biological compound are obtained by performing two or more of the following on the at least one known biological compound: liquid chromatography, mass spectrometry, ion mobility, tandem mass spectrometry, or
(iii) wherein the reference measurements for at least one known biological compound are predicted based on the plurality of reference measurements for at least one other known biological compound, or
any combination of the above.

3-4. (canceled)

5. The method according to claim 1, further comprising forming the two-dimensional plot prior to receiving the more than two sample measurements for the unknown biological sample.

6. The method according to claim 5, wherein forming the two-dimensional plot comprises one or more of the following for at least one of the known biological compounds:
(i) analysing the at least one of the known biological compounds with experimental devices to obtain the plurality of reference measurements for the at least one of the known biological compounds; and
calculating a reference point in two-dimension from the plurality of reference measurements for the at least one of the known biological compounds;
(ii) predicting the plurality of reference measurements for the at least one of the known biological compounds based on the plurality of reference measurements for at least one other known biological compound; and
calculating a reference point in two-dimension from the predicted plurality of reference measurements for the at least one of the known biological compounds;
(iii) categorizing the known biological compounds into multiple groups of isomers; and
categorizing the reference points into multiple groups of reference points corresponding to respective groups of isomers, wherein each reference point is categorized into the group of reference points corresponding to the group of isomers into which the corresponding known biological compound is categorized.

7. The method according to claim 6,
(i) wherein the experimental devices comprise two or more of the following: liquid chromatography, mass spectrometry, tandem mass spectrometry, ion mobility;
(ii) wherein predicting the plurality of reference measurements for the at least one of the known biological compounds comprises using a machine learning algorithm; and
(iii) wherein categorizing the known biological compounds into multiple groups of isomers comprises categorizing each known biological compound based on a mass value of the known biological compound.

8-11. (canceled)

12. The method according to claim 1,
(i) wherein each reference point is calculated by performing principal component analysis on the plurality of reference measurements; or
(ii) wherein calculating the sample point in the two-dimensional plot from the more than two sample measurements for the unknown biological sample comprises performing principal component analysis on the more than two sample measurements,
or a combination of the above.

13. The method according to claim 12, wherein performing principal component analysis on the plurality of reference measurements comprises:
transforming the plurality of reference measurements into a plurality of principal components,
(i) wherein the principal components are in an order such that variances of the principal components from a first principal component to a last principal component are in a descending order and each principal component is orthogonal to a next principal component in the order; and using the first and second principal components to form the reference point in two-dimension; or
(ii) wherein transforming the plurality of reference measurements into a plurality of principal components comprises calculating a plurality of principal component parameters and performing principal component analysis on the more than two sample measurements comprises using the plurality of principal component parameters;
or both (i) and (ii).

14-15. (canceled)

16. The method according to claim 13, wherein performing principal component analysis on the plurality of sample measurements comprises:
transforming the plurality of sample measurements into a plurality of principal components using the plurality of principal component parameters, wherein the principal components are in an order such that variances of the principal components from a first principal component to a last principal component are in a descending order and wherein each principal component is orthogonal to a next principal component in the order; and
using the first and second principal components to form the sample point in the two-dimensional plot.

17. The method according to claim 6, wherein identifying the unknown biological sample comprises one or more of:
(i) determining a reference point nearest to the sample point in the two-dimensional plot; and
identifying the unknown biological sample as the known biological compound corresponding to the determined nearest reference point; or
(ii) further comprises the following prior to determining the reference point nearest to the sample point in the two-dimensional plot:
categorizing the unknown biological sample into one of the multiple groups of isomers; and
retaining, in the two-dimensional plot, only the reference points in the group of reference points corresponding to the group of isomers into which the unknown biological sample is categorized; or
(iii) further comprises calculating an accuracy score based on a distance between the sample point and the determined nearest reference point.

18. (canceled)

19. The method according to claim 17,
(i) wherein the categorized reference points are reference points calculated from reference measurements obtained experimentally and the multiple groups of reference points form a first set of groups of reference points; and wherein the method further comprises categorizing reference points calculated from reference measurements obtained by a machine learning algorithm into a second set of groups of reference points corresponding to respective groups of isomers;
(ii) wherein categorizing the unknown biological sample into one of the groups of isomers comprises categorizing the unknown biological sample based on a mass to charge ratio value of the unknown biological sample, and
(iii) wherein the accuracy score comprises one of the following: a low confidence score, a medium confidence score, a high confidence score.

20. The method according to claim 19, further comprising the following if the unknown biological sample does not belong to any one of the multiple groups of isomers corresponding to the first set of groups of reference points:
categorizing the unknown biological sample into one of the groups of isomers corresponding to the second set of groups of reference points; and
determining the nearest reference point from the reference points in the group of reference points in the second set corresponding to the group of isomers into which the unknown biological sample is categorized.

21-23. (canceled)

24. The method according to claim 1,
(i) wherein the two-dimensional plot is formed from a first number of attributes; and wherein the method comprises using further plots, each further plot formed from a different number of attributes as compared to another plot; and/or
(ii) wherein the attribute of the unknown biological sample comprises one of the following: mass, mass to charge ratio, retention time, normalized retention time, glucose unit, collisional cross section, tandem mass spectrometry/mass spectrometry fragmentation, measured shift in retention time after exoglycosidase treatment, measured shift in mass to charge ratio after exoglycosidase treatment, measured shift in collisional cross section after exoglycosidase treatment, measured shift in tandem mass spectrometry/mass spectrometry fragmentation.

25. The method according to claim 24, wherein using further plots comprises performing the following for each further plot:
calculating a further sample point in the further plot based on at least one of the plurality of sample measurements for the unknown biological sample.

26. The method according to claim 1, wherein each reference point of the two-dimensional plot is calculated from:
(i) a first number of reference measurements for a first number of attributes of the corresponding known biological compound;
wherein the method further comprises calculating a sample point in each of a plurality of further plots based on at least one sample measurement for the unknown biological sample;
wherein each of the plurality of further plots comprises a plurality of stored reference points corresponding to respective known biological compounds, each reference point of the further plot calculated from at least one reference measurement for at least one attribute of the corresponding known biological compound; and
wherein for each further plot, the number of attributes from which the reference points are calculated differ from the first number and differ from the number of attributes from which the reference points in a different further plot are calculated; or
(ii) from three reference measurements for three attributes of the corresponding known biological compound and wherein the method further comprises:
calculating a second sample point in a second two-dimensional plot based on two sample measurements for the unknown biological sample, wherein the second two-dimensional plot comprises a plurality of stored reference points corresponding to respective known biological compounds, each reference point of the second two-dimensional plot calculated from two reference measurements for two attributes of the corresponding known biological compound; and
calculating a third sample point in a third plot based on one sample measurement for the unknown biological sample, wherein the third plot comprises a plurality of stored reference points corresponding to respective known biological compounds, each reference point of the third plot calculated from one reference measurement for one attribute of the corresponding known biological compound.

27-29. (canceled)

30. The method according to claim 26, wherein the method further comprises:
for each plot, determining a reference point nearest to the sample point in the plot; and
identifying the unknown biological sample as the known biological compound corresponding to the most number of determined nearest reference points.

31-32. (canceled)

33. The method according to claim 1, wherein the unknown biological sample comprises one of the following: glycan, metabolite, antibody.

34. The method according to claim 33, wherein the glycan comprises one or more of the following: glycospingolipid glycan, N-glycan, O-glycan, and procainamide-labelled glycan.

35. (canceled)

36. A computer program product comprising computer-readable instructions that implement an application for identifying an unknown biological sample, wherein the computer program product is configured to be executed on one or more computing devices, each having one or more processors:
wherein the application is configured to provide a two-dimensional plot comprising a plurality of stored reference points corresponding to respective known biological compounds and wherein each reference point is calculated from a plurality of reference measurements for more than two attributes of the corresponding known biological compound, each attribute being different from another attribute; and
wherein the application comprises instructions for:
receiving more than two sample measurements for the unknown biological sample, each sample measurement for an attribute of the unknown biological sample;
calculating a sample point in a two-dimensional plot from the more than two sample measurements for the unknown biological sample; and
identifying the unknown biological sample by comparing the sample point against the plurality of reference points in the two-dimensional plot.

37. A kit comprising:
an extraction device for extracting an unknown biological sample;
at least one experimental device for determining sample measurements for the extracted unknown biological sample; and
a computing device configured to execute the computer program product according to claim 36.

38. An apparatus comprising:
a memory; and
at least one processor coupled to the memory and configured to:
receive more than two sample measurements for an unknown biological sample, each sample measurement for an attribute of the unknown biological sample;
calculate a sample point in a two-dimensional plot from the more than two sample measurements for the unknown biological sample;
wherein the two-dimensional plot comprises a plurality of stored reference points corresponding to respective known biological compounds and wherein each reference point is calculated from a plurality of reference measurements for more than two attributes of the corresponding known biological compound, each attribute being different from another attribute; and
identify the unknown biological sample by comparing the sample point against the plurality of reference points in the two-dimensional plot.
